(19) United States
(12) Patent Application Publication
(10) Pub. No.: US 2007/0014739 A1
(43) Pub. Date: Jan. 18, 2007

(54) Compositions and methods for controlling biofilms and bacterial infections

(76) Inventors: Gary R. Eldridge, Encinitas, CA (US); Matt Goering, Encinitas, CA (US)

Correspondence Address: THOMPSON COBURN, LLP, ONE US BANK PLAZA, SUITE 3500, ST LOUIS, MO, 63101, US

(21) Appl. No.: 11/181,556

(22) Filed: Jul. 14, 2005

(51) Int. Cl. A61K 8/97

(52) U.S. Cl. 424/49; 424/58

(57) ABSTRACT

The present invention provides compounds and compositions useful for controlling bacterial biofilms as well as for controlling and/or preventing bacterial infections. The compounds of the invention are pentacyclic acid triterpenes. Methods for controlling biofilms and for controlling and/or preventing bacterial infections are also disclosed.

### CROSS REFERENCE TO RELATED APPLICATIONS

This application claims priority from U.S. patent application Ser. No. 11/085,279, filed on Mar. 21, 2005, which claimed priority to U.S. provisional application Ser. Nos. 60/587,680 (filed on Jul. 14, 2004) and 60/609,763 (filed on Sep. 14, 2004). The contents of U.S. patent application Ser. No. 11/085,279, U.S. provisional Application Ser. No. 60/587,680 and U.S. provisional Application Ser. No. 60/609,763 are incorporated herein in their entirety.

### FIELD OF THE INVENTION

The present invention generally relates to compounds useful for reducing or preventing formation of a biofilm. The present invention also relates to compounds useful for reducing or preventing the formation of a biofilm in a tissue and for controlling, preventing or treating a chronic bacterial infection.

### BACKGROUND

Bacterial biofilms exist in natural, medical, and engineering environments. The biofilms offer a selective advantage to a microorganism to ensure its survival, or allow it a certain amount of time to exist in a dormant state until suitable growth conditions arise. Unfortunately, this selective advantage poses serious threats to animal health, especially human health.

Chronic infections involving biofilms are serious medical problems throughout the world. For example, biofilms are involved in 65% of human bacterial infections. Biofilms are involved in prostatitis, biliary tract infections, urinary tract infections, cystitis, lung infections, sinus infections, ear infections, acne, rosacea, dental caries, periodontitis, nosocomial infections, open wounds, and chronic wounds.

Compounds that modify biofilm formation would have a substantial medical impact by treating many chronic infections, reducing catheter- and medical device-related infections, and treating lung and ear infections. The potential market for biofilm inhibitors could be enormous given the sheer number of cases in which biofilms contribute to the medical problems. The inhibitors may be used to cure, treat, or prevent a variety of conditions, such as, but are not limited to, arterial damage, gastritis, urinary tract infections, pyelonephritis, cystitis, otitis media, otitis extema, leprosy, tuberculosis, benign prostatic hyperplasia, chronic prostatitis, chronic lung infections of humans with cystic fibrosis, osteomyelitis, bloodstream infections, skin infections, open or chronic wound infections, cirrhosis, and any other acute or chronic infection that involves or possesses a biofilm.

In the United States, the market for antibiotics is greater than $8.5 billion. After cardiovascular therapeutics, the sales of antibiotics are the second largest drug market in the United States. The antibiotic market is fueled by the continued increase in resistance to conventional antibiotics. Approximately 70% of bacteria found in hospitals resist at least one of the most commonly prescribed antibiotics. Because biofilms appear to reduce or prevent the efficacy of antibiotics, co-administration of biofilm inhibitors could significantly boost the antibiotic market.

Using the protection of biofilms, microbes can resist antibiotics at a concentration ranging from 1 to 1.5 thousand times higher than the amount used in conventional antibiotic therapy. During an infection, bacteria surrounded by biofilms are rarely resolved by the immune defense mechanisms of the host. It has been proposed that in a chronic infection, a biofilm gives bacteria a selective advantage by reducing the penetration of an antibiotic into the depths of the tissue needed to completely eradicate the bacteria'ds existence (Costerton J W et al., Science. 1999 May 21;284(5418):1318-22).

Traditionally, antibiotics are discovered using the susceptibility test methods established by the National Committee for Clinical Laboratory Standards (NCCLS). The methods identify compounds that specifically affect growth or death of bacteria. These methods involve inoculation of a bacterial species into a suitable growth medium, followed by the addition of a test compound, and then plot of the bacterial growth over a time period post-incubation. Unfortunately these antibiotics derived from the NCCLS methods would not be effective therapeutics against chronic infections involving biofilms because the methods do not test compounds against bacteria in a preformed biofilm. Consistently, numerous publications have reported a difference in gene transcription in bacteria living in biofilms from bacteria in suspension, which further explains the failure of conventional antibiotics to eradicate biofilm infections (Sauer, K. et al. J. Bacteriol. 2001, 183:6579-6589).

Biofilm inhibitors can provide an alternative treatment approach for certain infections. Biofilm inhibitors, on the other hand, act on the biological mechanisms that provide bacteria protection from antibiotics and from a host's immune system. Biofilm inhibitors may be used to “clear the way” for the antibiotics to penetrate the affected cells and eradicate the infection. Traditionally, treatment of nosocomial infections requires an administration of a combination of products, such as amoxicillin/clavulanate and quinupristin/dalfopristin, or an administration of two antibiotics simultaneously. In one study of urinary catheters, rifampin was unable to eradicate methicillin-resistant Staphylococcus aureus in a biofilm but was effective against planktonic, or suspended cells (Jones, S. M., et. al., “Effect of vancomycin and rifampicin on methicillin-resistant Staphylococcus aureus biofilms”, Lancet. 357:40-41, 2001).

Bacteria have no known resistance to biofilm inhibitors. Biofilm inhibitors are not likely to trigger growth-resistance mechanisms or affect the growth of the normal human flora. Thus, biofilm inhibitors could potentially extend the product life of antibiotics.

Biofilm inhibitors can also be employed for the treatment of acne. Acne vulgaris is the most common cutaneous disorder. Propionibacterium acnes, is the predominant microorganism present in acne. The bacteria reside in biofilms. The bacteria's existence in a biofilm matrix provides them with a protective, physical barrier that limits the effectiveness of antimicrobial agents (Burkhart, C. N. et. al., “Microbiology's principle of biofilms as a major factor in the pathogenesis of acne vulgaris”, International J. of Dermatology. 42:925-927, 2003). Biofilm inhibitors may be used to effectively prevent, control, reduce, or eradicate P. acnes biofilms in acne.

Plaque biofilms contribute to cavities and periodontitis. Plaque biofilms accumulate due to bacterial colonization of Streptococci spp., such as S. mutans, S. sobrinas, S. gordonii, and Porphyromonas gingivalis, and Actinomyces spp (Demuth, D. et al. Discrete Protein Determinant Directs the Species-Species Adherence of Porphyromonas gingivalis to Oral Streptococci, Infection and Immunity, 2001, 69(9) p5736-5741; Xie, H., et al. Intergeneric Communication in Dental Plaque Biofilms. J. Bacteriol. 2000, 182(24), p7067-7069). The primary colonizing bacteria of plaque accumulation are Streptococci spp., while P. gingivalis are a leading cause of periodontitis (Demuth, D. et al. Discrete Protein Determinant Directs the Species-Species Adherence of Porphyromonas gingivalis to Oral Streptococci, Infection and Immunity, 2001, 69(9) p5736-5741). Biofilm inhibitors can be employed to prevent microorganisms from adhering to surfaces that may be porous, soft, hard, semi-soft, semi-hard, regenerating, or non-regenerating. These surfaces may be teeth, polyurethane material of central venous catheters, or metal, alloy, or polymeric surfaces of medical devices, or regenerating proteins of cellular membranes of mammals. These inhibitors can be coated on or impregnated into these surfaces at a concentration sufficient to control, reduce, or eradicate the microorganisms adherence to these surfaces.

Chronic wound infection represents another illness that is difficult to eradicate. Examples of the most common types of chronic wounds are diabetic foot ulcers, venous leg ulcers, arterial leg ulcers, and pressure ulcers. Diabetic foot ulcers appear to be the most prevalent. These wounds are typically colonized by multiple species of bacteria including Staphylococcus spp., Streptococcus spp., Pseudomonas spp. and Gram-negative bacilli (Lipsky, B. Medical Treatment of Diabetic Foot Infections. Clin. Infect. Dis. 2004, 39, p.S104-14). Based on clinical evidence, microorganisms cause or contribute to chronic wound infections. Only recently have biofilms been implicated in these infections (Harrison-Balestra, C. et al. A Wound-isolated Pseudomonas aeruginosa Grow a Biofilm In Vitro Within 10 Hours and Is Visualized by Light Microscopy, Dermatol Surg 2003, 29; 631-635; Edwards, R. et al. Bacteria and wound healing. Curr Opin Infect Dis, 2004, 17; 91-96). Approximately 140,000 amputations occur each year in the United States due to chronic wound infections that could not be treated with conventional antibiotics. Unfortunately, treating these infections with high doses of antibiotics over long periods of time contributes to the development of antibiotic resistance (Howell-Jones, R. S., et al. A review of the microbiology, antibiotic usage and resistance in chronic skin wounds. J. Antimicrob. Ther. January 2005). Biofilm inhibitors in a combination therapy with antibiotics may provide an effective alternative to the treatment of chronic wounds.

Recent publications describe the cycles of the pathogenesis of numerous species of bacteria involving biofilms. For example, Escherichia coli, which causes recurrent urinary tract infections, undergo a cycle of binding to and then invading a host's bladder epithelial cells. The E. coli form a biofilm intracellularly, modify its morphology, and then burst out of the host cells to repeat the cycle of pathogenesis (Justice, S. et al. Differentiation and development pathways of uropathogenic Escherichia coli in urinary tract pathogenesis, PNAS 2004, 101(5): 1333-1338). The authors suggest that this repetitive cycle of pathogenesis of E. coli may explain the recurrence of the infection.

In 1997, Finlay, B. et al. reported that numerous bacteria, including Staphylococci, Streptococci, Bordetella pertussis, Neisseria spp., Helicobactor pylori, and Yersinia spp., adhere to mammalian cells during their pathogenesis. The authors hypothesized that the adherence would lead to an invasion of the host cell. Later publications confirm this hypothesis (Cossart, P. Science, 2004, 304; 242-248; see additional references infra). Other publications presented similar hypotheses to Mulvey, M. et al. (Mulvey, M. et al. “Induction and Evasion of Host Defenses by Type 1-Piliated Uropathogenic E. coli ” Science 1998, 282 p. 1494-1497). In particular, Mulvey, M. et al. stated invasion of E. coli into epithelial cells provide protection from the host's immune response to allow a build up of a large bacterial population.

Cellular invasion and biofilm formation appear to be integral to the pathogenesis of most, if not all bacteria. Pseudomonas aeruginosa have been shown to invade epithelial cells during lung infections (Leroy-Dudal, J. et al. Microbes and Infection, 2004, 6, p. 875-881). P. aeruginosa are the principal infectious organisms found in the lungs of cystic fibrosis patients, and the bacteria exist within a biofilm. Antibiotics like tobramcyin, and other current antibacterial compounds, do not provide effective treatment against biofilms of chronic infections, perhaps because antibiotic therapy fails to eradicate the biofilm.

The pathogenesis of cellular invasion and biofilm formation gram-negative bacteria follow conserved mechanisms. For example, Haemophilus influenzae invade epithelial cells and form biofilms (Hardy, G. et al., Methods Mol. Med., 2003, 71; 1-18; Greiner, L. et al., Infection and Immunity, 2004, 72(7); 4249-4260). Burkholderia spp. invade epithelial cells and form biofilm (Utaisincharoen, P. et al., Microb Pathog. 2005, 38(2-3); 107-112; Schwab, U. et al. Infection and Immunity, 2003, 71(11); 6607-6609). Klebsiella pneumoniae invade epithelial cells and form biofilm (Cortes, G et al. Infection and Immunity. 2002, 70(3); 1075-1080; Lavender, H. et al., Infection and Immunity. 2004, 72(8); 4888-4890). Salmonella spp. invade epithelial cells and form biofilms (Cossart, P. Science, 2004, 304; 242-248; Boddicker, J. et al., Mol. Microbiol. 2002, 45(5); 1255-1265). Yersinia pestis invade epithelial cells and form biofilms (Cossart, P. Science, 2004, 304; 242-248; Jarrett, C. et al. J. Infect. Dis., 2004, 190; 783-792). Neisseria gonorrhea invade epithelial cells and form biofilms (Edwards, J. et al., Cellular Micro., 2002, 4(9); 585-598; Greiner, L. et al., Infection and Immunity. 2004, 73(4); 1964-1970). Burkholderia sp. are another important class of gram-negative bacterial pathogens. Chlamydia sp., including Chlamydia pneumoniae is an intracellular, Gram-negative pathogen implicated in respiratory infections and chronic diseases such as atherosclerosis and Alzheimer's disease (Little, C. S. et al., Infection and Immunity. 2005, 73(3); 1723-34).

These Gram-negative bacteria cause lung, ear, and sinus infections, gonorrhoeae, plague, diarrhea, typhoid fever, and other infectious diseases. E. coli and P. aeruginosa are two of the most widely studied Gram-negative pathogens. Researchers believe that the pathogenesis of these bacteria involves invasion of host cells and formation of biofilms. These models have enabled those skilled in the art to understand the pathogenesis of other Gram-negative bacteria.

Gram-positive bacteria also share conserved mechanisms of bacterial pathogenesis involving cellular invasion and biofilm formation. Staphylococcus aureus invade epithelial cells and form biofilms (Menzies, B. et al., Curr Opin Infect Dis, 2003, 16; 225-229; Ando, E. et al., Acta Med Okayama, 2004, 58(4); 207-14). Streptococcus pyogenes invade epithelial cells and form biofilms (Cywes, C. et al., Nature, 2001,414; 648-652; Conley, J. et al., J. Clin. Micro., 2003, 41(9); 4043-4048).

U.S. Pat. No. 4,606,911 (referred to as the '911 patent hereafter) describes compounds that selectively inhibit the growth and anti-adherence activities of Gram-positive mouth bacteria Streptococcus mutans but do not effect other bacteria. This patent discloses the use of oleanolic and ursolic acid as inhibiting the growth of S. mutans and promoting anti-adherence activities. The patent also lists compositions for oral care products in the claims. However, the patent clearly states the benefit of ursolic acid and related compounds is that they do not affect oral microorganisms other than S. mutans. Growth inhibition data presented in this patent indicated that ursolic acid completely inhibited S. mutans and S. salivaris (both gram-positive Streptococcal bacteria) yet failed to inhibit the gram-positive bacterium S. aureus (gram-positive) or the gram negative bacteria E. coli and P. aeruginosa. Oleanolic acid displayed incomplete inhibition of S. mutans and S. salivaris (both gram-positive bacteria) yet failed to inhibit the gram-positive bacterium S. aureus or the gram negative bacteria E. coli and P. aeruginosa. The '911 patent thus teaches that these compounds are useful for treating tooth decay by specifically inhibiting S. mutans growth and adherence. Consequently, the '911 patent neither demonstrates nor suggests that ursolic acid and oleanolic acid or the derivatives described herein prevent, inhibit, or reduce the in vitro or in vivo formation of biofilms. Furthermore, the '911 patent neither demonstrates nor suggests that ursolic acid and oleanolic acid can prevent or treat bacterial infections caused by microorganisms other than S. mutans. Moreover, the '911 patent does not teach or suggest use of ursolic acid and oleanolic acid in oral care products in combination with an antimicrobial agent or antibiotic. As demonstrated in the examples, the compounds of this instant invention may be used in combination with antibiotics to treat chronic infections like plaque.

Centella asiatica plant extracts have been used to treat dermatological conditions, wounds, burns and reportedly reduce of scar formation (Kartnig, T in Herbs, Spices and Medicinal Plants, vol3., Eds. Cracker, L. E. and Simon, J. E., Oryx Press, Arizona, USA, 1998, pp. 145-173). Centella asiatica extracts have also been reported to be used to treat/alleviate eruptive disease, abdominal pain, dysentery, jaundice due to damp heat, urinary calculi, hematuria, vomiting, epistaxis, ocular irritation, pharyngitis, wind rash, scabies, toxic swelling, as well as internal and external trauma (Oriental Materia Medica: A Concise Guide, Keats Publishing, Inc., New Canaan, Conn., 1986, pp. 443-444). The preparations of crude plant extracts contain varying amounts of asiaticoside, madecassoside, brahmoside, brahminoside, asiatic acid, and madecassic acid. For example, Syntex Research Centre marketed a titrated plant extract of Centella asiatica for the treatment of burns that contained, among other compounds, asiatic acid, madecassic acid, and asiaticoside. Researchers have tried unsuccessfully to determine the identity of the compounds in the extract that provide the therapeutic benefit. Although it has been proposed that the triterpenoid compounds stimulate collagen synthesis, published laboratory results contradict this hypothesis (Coldren C. et al. (2002) Planta Med 69, 725-732). It is also reported that water extracts of Centella, which are expected to contain triterpene glycosides rather than triterpenes, have antibacterial activity (Oriental Materia Medica: A Concise Guide, 1986; Keats Publishing, Inc., New Canaan, Conn.; pp. 443-444). None of these references suggest the use of asiatic and madecassic acid, just two of the many constituents of the Centella asiatica extracts, as biofilm or bacterial infection inhibitors.

Accordingly, for the reasons discussed above and others, there exists an unmet need for compounds that serve as biofilm inhibitors and/or that would be useful for preventing, reducing, or inhibiting bacterial infections.

### SUMMARY OF INVENTION

The present invention provides a compositions comprising an antimicrobial agent or biocide, a pentacyclic acid triterpene compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, 3-acetyl oleanolic acid and Caulophyllogenin and an acceptable carrier. Salts, hydrates, solvates, prodrugs and N-oxides of the pentacyclic acid triterpene compounds listed above are also contemplated by the present invention. As demonstrated herein, such compositions are useful in controlling bacterial infections and/or biofilm formation in a variety of subjects including animals such as mammals and human patients as well as plants.

This invention also provides methods for preventing, inhibiting or reducing a biofilm comprising contacting the biofilm or a cell capable of biofilm formation with an effective amount of a composition or a compound comprising a pentacyclic acid triterpene compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, 3-acetyl oleanolic acid and Caulophyllogenin and an acceptable carrier. Inhibition or reduction of biofilm formation may be effected either in vivo or in vitro. Compositions used to inhibit, reduce or prevent biofilm formation may further include either an antimicrobial agent, antibiotic or a biocide. The methods also provide for preventing, inhibiting or reducing biofilm formation on a variety of substrates.

This invention further provides for methods of inhibiting or preventing a bacterial infection in a subject by administering an effective amount of a composition comprising a pentacyclic acid triterpene compound selected from the group consisting of 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid and Caulophyllogenin and an acceptable carrier. The subject may be a human, an animal or a plant. When the subject is an mammal or a human, the carrier is a pharmaceutically acceptable carrier. When the subject is a plant, the carrier is an agriculturally acceptable carrier

The invention finally provides for processes of making the compositions described herein. Such processes involve combining an essentially pure preparation of an antimicrobial agent, an essentially pure preparation of a pentacyclic acid triterpene compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof and an acceptable carrier. Such compositions may be either pharmaceutical compositions, in which case a pharmaceutically acceptable carrier is used, or agricultural compositions, in which case an agriculturally acceptable carrier is used. The essentially pure antimicrobial agent or pentacyclic acid triterpene compound can be obtained from any one of a commercial source, a natural source, or by direct synthesis.

### BRIEF DESCRIPTION OF THE DRAWINGS

FIG. 1 shows a confocal microscopy image of an IBC of E coli from a bladder of a control mouse inoculated with E. coli UT189 (Untreated) and a confocal microscopy image of a small collection of E coli from a bladder of a mouse inoculated with E. coli UTI89 and corosolic acid (Treated).

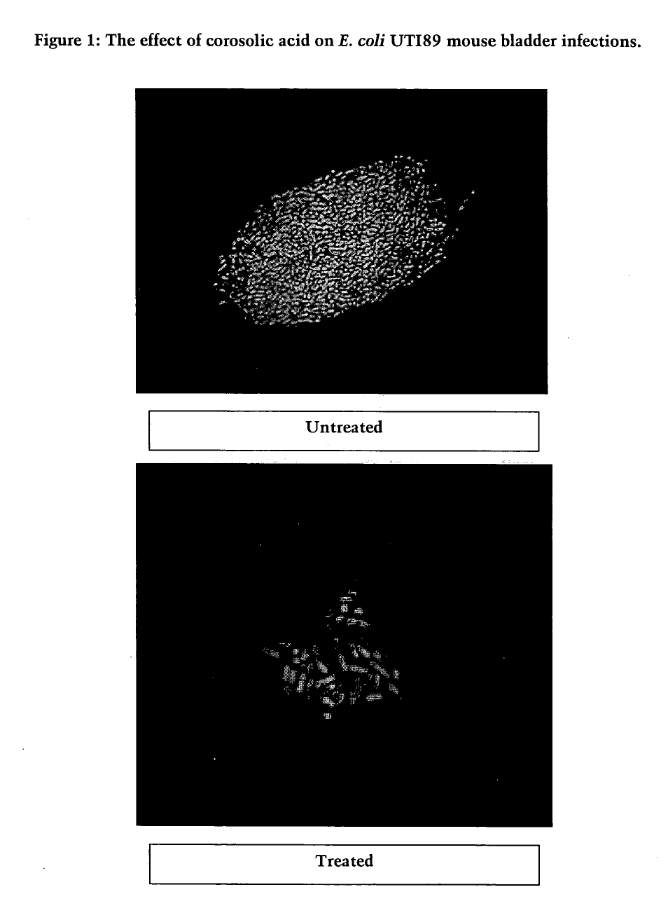

FIG. 2 shows the Ursane and Oleanane scaffold structures with Carbon number designations.

### DESCRIPTION OF THE INVENTION

Definitions:

“Acceptable carrier” refers to a carrier that is not deleterious to the other ingredients of the composition and is not deleterious to material to which it is to be applied. “Pharmaceutically acceptable carrier” refers to a carrier that is not deleterious to the other ingredients of the composition and is not deleterious to the human or other animal recipient thereof. “Agriculturally acceptable carrier” refers to a carrier that is not deleterious to the other ingredients of the composition and is not deleterious to the plant recipient thereof. In the context of the other ingredients of the composition, “not deleterious” means that the carrier will not react with or degrade the other ingredients or otherwise interfere with their efficacy. Interference with the efficacy of an ingredient does not encompass mere dilution of the ingredient. In the context of the animal or plant host, “not deleterious” means that the carrier is not injurious or lethal to the plant or animal.

“Administration” refers to any means of providing a compound or composition to a subject. Non-limiting examples of administration means include oral, topical, rectal, percutaneous, parenteral injection, intranasal and inhalation delivery.

“Biofilm” refers to an extracellular matrix in which microorganisms are dispersed and/or form colonies. The biofilm typically is made of polysaccharides and other macromolecules.

“Commercial source” refers to a vendor that provides the desired compound.

“Direct synthesis” refers to production of the desired compound by reacting appropriate compound precursors under appropriate conditions to obtain the desired compound.

“Effective amount” refers to the amount of compound or composition that, in the case of biofilm formation, will reduce the size or volume of existing biofilms; reduce the rate at which bacteria are capable of producing biofilm; or will inhibit or prevent the formation of biofilm by one or more microorganisms. In the context of treating a bacterial infection, an “effective amount” refers the amount of a compound or composition that will reduce the degree of an existing infection or will inhibit or prevent an infection from occurring.

“Essentially pure preparation” refers to a preparation in which the concentration of the desired ingredient is at least 95% or more of the preparation by weight. In the context of this processes used in this invention, the antimicrobial agents and pentacyclic acid triterpene compounds typically and preferably make up 99% or more by weight of the preparation and are referred to herein as “highly pure” preparations.

“In vivo”, in the context of biofilm formation, refers to effects mediated in or upon living organisms or subjects. Effects mediated on biofilms associated with medical devices such as central venous catheters, urinary catheters, endotracheal tubes, mechanical heart valves, pacemakers, vascular grafts, stents, and prosthetic joints located within a living organism or subject are considered as “in vivo” uses of the compounds and compositions described herein.

“In vitro”, in the context of biofilm formation, refers to effects mediated on substrates located outside of an organism that are potential sites of biofilm formation. Non-limiting examples of substrates include vessel hulls, cars, airplanes, industrial equipment, devices, membranes, filters, microtiter plates, continuous flow chambers, bioreactors, fermentors, chemostats and machinery.

“Is one that permits” as it relates to a pharmaceutically acceptable carrier that has characteristics that enable the preparation to be used for a given mode of administration of the composition. For example, pharmaceutically acceptable carriers that permit parenteral administration to an animal are liquids that are not injurious or lethal to the animals when so injected. Such carriers often comprise sterile water, which may be supplemented with various solutes to increase solubility. Sterile water or sterile water supplemented with solutes is thus a pharmaceutically acceptable carrier that permits parental administration.

“Natural source” is defined as any living organism or material derived therefrom. Note that in the context of this application, the natural source may be a non-naturally occurring living organism or material derived therefrom.

“Reducing or inhibiting” in reference to a biofilm refers to the prevention of biofilm formation or growth, a reduction in the rate of biofilm formation or growth, reduction or removal of preformed or existing biofilm, as well as the partial or complete inhibition of biofilm formation or growth.

“Subject in need thereof” refers to living organism that would benefit from either prevention or reductions in the degree of a bacterial infection. Subjects may include animals or more specifically, mammals or humans. Subjects may also include plants.

“Substrate” refers to any material to which the compound or a composition containing the compound may be applied.

Compounds Used in the Invention

In accordance with the present invention, it has been found that a group of pentacyclic acid triterpene compounds is surprisingly effective in inhibiting the formation of biofilms, reducing existing biofilms and inhibiting bacterial infections. The biofilm inhibiting activity of the pentacyclic acid triterpenes was previously unappreciated. Since the pentacyclic acid triterpenes shown herein to inhibit biofilm formation do not directly inhibit the growth of many bacteria outside of an infected host, the use of the pentacyclic acid triterpene compounds in inhibiting the growth of those same bacteria in an infected host was also unappreciated. Furthermore, it has also surprisingly been shown that the co-administration of pentacyclic acid triterpene compound with an antimicrobial agent or antibiotic to a bacterial biofilm provides increased susceptibility of the bacteria to the antibiotic. The instant invention thus provides for compositions comprising both pentacyclic acid triterpenes and antimicrobial agents or antibiotics, methods of using compositions comprising both pentacyclic acid triterpenes and antimicrobial agents or antibiotics, and processes for making the compositions comprising both pentacyclic acid triterpenes and antimicrobial agents or antibiotics.

A key feature of this invention is that it further provides methods for obtaining the pentacyclic acid triterpene compounds at dosage amounts, concentrations and purity levels that permit effective inhibition of biofilm formation or bacterial infections. Although crude plant extracts containing pentacyclic acid triterpene compounds along with a variety of other compounds have been used for a variety of indications, it is not anticipated that those crude extracts would provide result-effective dosages of the pentacyclic acid triterpene for use as biofilm or bacterial infection prevention or inhibition as taught herein. Furthermore, it is also anticipated that providing the pentacyclic acid triterpene in purified form will eliminate potential side effects associated with the administration of crude plant extracts containing a variety of compounds. Finally, it is also anticipated that providing the pentacyclic acid triterpene in purified form will enable consistent dosing of subjects and thus result in more consistent inhibition or prevention of biofilm formation or bacterial infections. By identifying pentacyclic acid triterpenes as a key biofilm and bacterial infection inhibiting component of crude plant extracts and providing means for obtaining the pentacyclic acid triterpenes in a pure and concentrated form, the instant invention thus provides for effective biofilm and bacterial infection inhibiting compounds, compositions and methods of use.

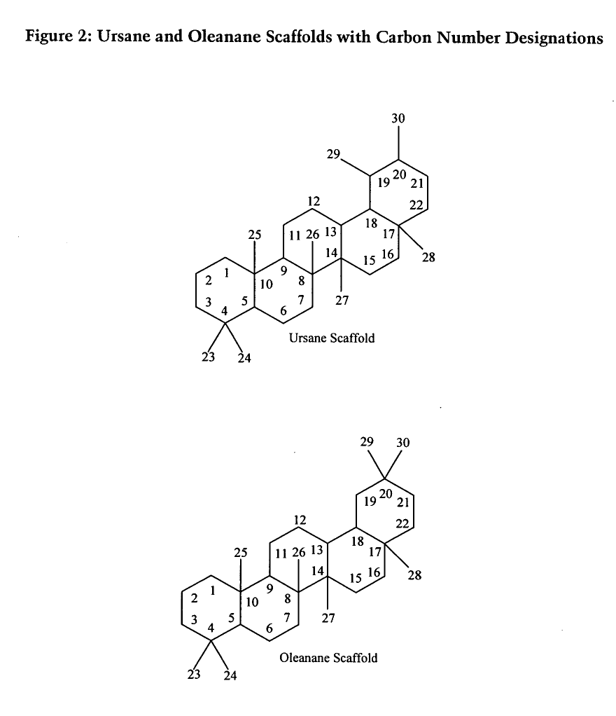

The broad group of compounds useful in the practice of this invention are collectively referred to herein as pentacyclic acid triterpenes. Pentacyclic acid triterpenes are defined in the context of this invention to encompass any compounds that have either the ursane or oleanane triterpene scaffolds depicted below and in FIG. 2 wherein C28 is a carboxylic acid. More preferably, these compounds will have a carboxylic acid at position 28, a single, unsubstituted methyl at positions 25, 26, 27, 29, and 30 and a single unsubstituted or substituted methyl at positions 23 and 24.

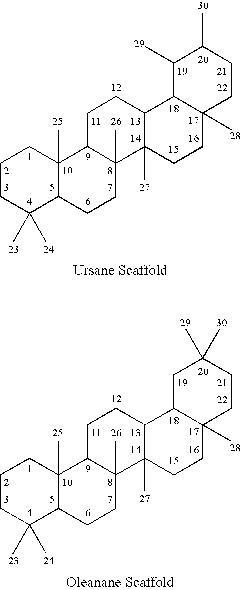

The following exemplary pentacyclic acid triterpene compounds have been shown to prevent or inhibit biofilms and/or to prevent or inhibit bacterial infections in ce with the present invention:

Compound 99 (30-hydroxyursolic acid):

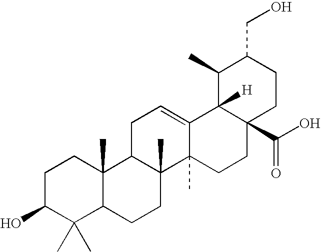

Compound 107 (2-hydroxyoleanic acid):

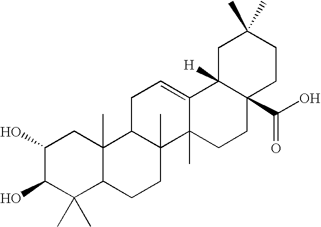

Compound 108 (Corosolic acid):

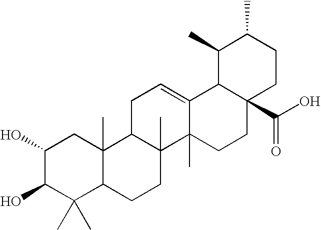

Compound 110 (Ursolic acid):

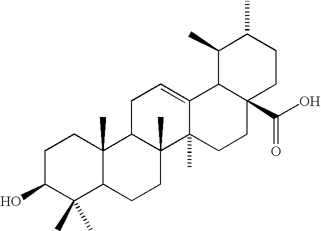

Compound 116 (−3-O-[3-hydroxy, 4-methoxy-cinnamoyl(trans-)]-2hydroxyursolic acid):

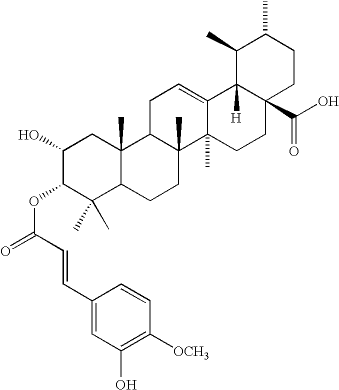

Compound 188 (3-[4-Hydroxycinnamoyl (cis-)], 20-hydroxy-ursolic acid):

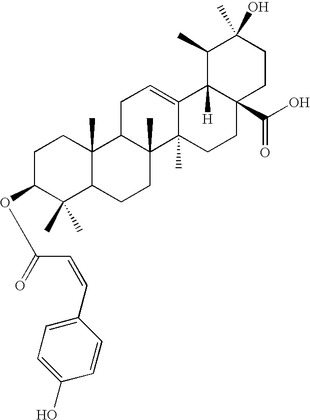

Compound 189 (3-[4-hydroxycinnamoyl(trans-)]-2-hydroxyursolic acid):

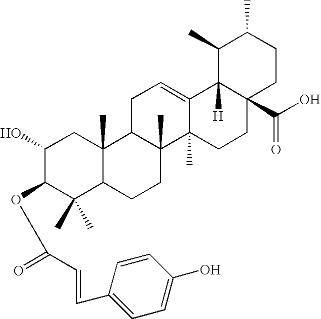

Compound 190 (3-[4-hydroxycinnamoyl(cis-)]-2-hydroxyursolic acid):

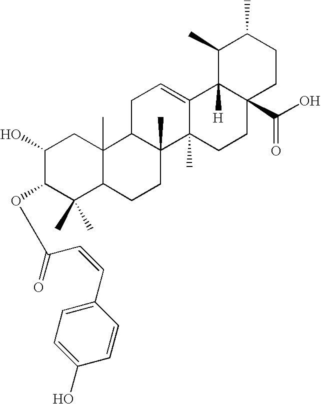

Compound 192 (Euscaphic acid):

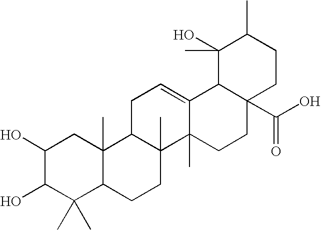

Compound 195 (20B-hydroxyursolic acid):

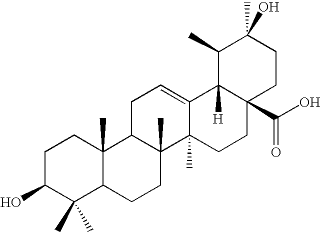

Compound 203 (Tormentic acid):

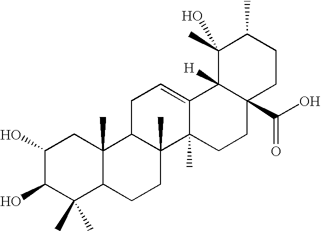

Compound 225 (Oleanolic acid):

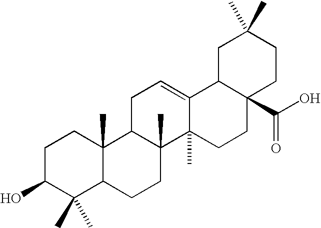

Compound 255 (Asiatic acid):

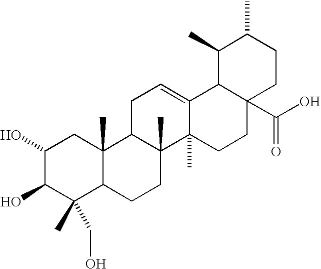

Compound 314 (Madecassic acid):

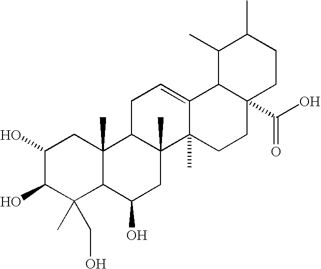

Compound 323 (Caulophyllogenin):

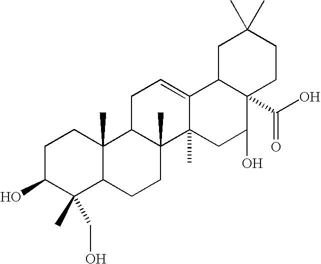

Compound 456 (Pygenic Acid A):

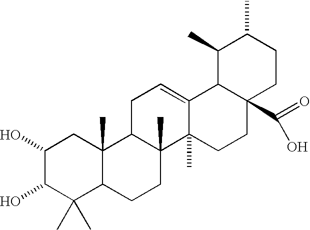

Compound 430 (Pygenic Acid B):

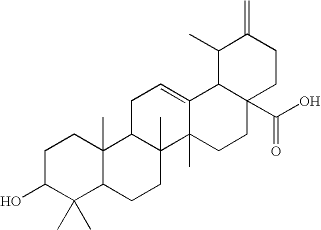

Compound 455 (Echinocystic Acid):

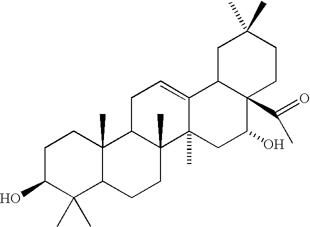

Compound 480 (3-acetyl oleanolic acid)

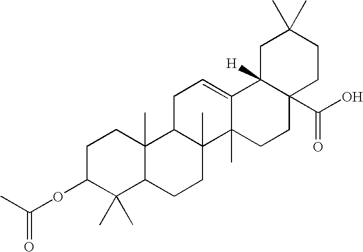

The compounds described herein to be useful in practicing the invention contain one or more asymmetric centers and thus give rise to enantiomers, diastereomers, and other stereoisomeric forms that may be defined, in terms of absolute stereochemistry, as (R)- or (S)-. The present invention encompasses all such possible isomers, as well as their racemic and optically pure forms. Optical isomers may be prepared from their respective optically active precursors using the procedures described herein, or by resolving the racemic mixtures. The resolution can be carried out in the presence of a resolving agent, by chromatography or by repeated crystallization or by some combination of these techniques which are known to those skilled in the art.

When the compounds described herein contain olefinic double bonds, other unsaturation, or other centers of geometric asymmetry, and unless specified otherwise, it is intended that the compounds include both E and Z geometric isomers or cis- and trans-isomers. Similarly, all tautomeric forms are intended to be encompassed by the present invention. The cis-trans configuration relative to any double bond appearing herein is selected for convenience only and is not intended to designate a particular configuration unless the text so states; thus, the cis or trans configuration is depicted arbitrarily herein and notwithstanding the configuration shown, may be cis, trans, or a mixture of the two in any proportion.

Methods of Isolation, Purification, and Modification

The pentacyclic acid triterpene compounds disclosed herein may be obtained from either natural sources, direct synthesis or purchased from commercial vendors. Furthermore, the pentacyclic acid triterpene compounds obtainable from any of the sources described may be separated and purified using methods such as column chromatography, high pressure liquid chromatography, and/or recrystallization. As will be appreciated by the skilled artisan, further methods of synthetically producing and derivatizing the compounds disclosed herein will be evident to those of ordinary skill in the art. Additionally, the various isolation, purification, and/or synthetic steps may be performed in an alternate sequence or order to produce the desired compounds.

Many of the compounds described herein may be isolated and purified from a natural source such as plants or materials derived from plants. Table 1 shows the representative plant species from which various compounds described herein have been isolated and purified. Those of ordinary skill in the art will appreciate that the compounds listed in Table 1 may be found in and isolated from other varieties within the respective plant family, genus, etc. represented therein. Using the procedures described herein or routine modifications of the procedures, a skilled artisan is taught how to isolate the pentacyclic acid triterpene compounds described herein. Structurally related pentacyclic triterpene compounds may also be isolated from plants known to contain these compounds or from plants that may not be known to contain these compounds by these procedures or modifications thereof. It is also contemplated that the compounds could be isolated from a natural source such as plant cells grown in culture or from media derived from plant cells grown in culture. Another natural source of the compounds might be root exudates harvested from hydroponically grown plants. Finally, the plant used as a “natural source” for the compounds need not be a naturally occurring plant but rather might be a cultivated or a transgenic variety of plant. However, using the procedures described herein a skilled artisan is taught how to isolate structurally related compounds previously identified in publications of which no known biofilm inhibition activities were anticipated, but now become evident because of the written descriptions contained herein.

TABLE ONE
Plant Sources of Pentacyclic Acid Triterpene Compounds

| | Compound | |
|---|---|---|
| Compound | Number | Plant Species |
| 30-hydroxyursolic acid | 99 | Arctostaphylos tomentosa |
| | | (California, USA); Arctostaphylos |
| | | edmundsii (California, USA); and |
| | | Phyla nodiflora (Texas, USA) |
| 2-hydroxyoleanolic acid | 107 | Diospyros dendo (Gabon, Africa) |
| Corosolic acid | 108 | Diospyros dendo (Gabon, Africa) |
| Ursolic acid | 110 | Diospyros dendo (Gabon, |
| | | Africa); Arctostaphylos |
| | | tomentosa (California, USA); |
| | | Arctostaphylos edmundsii |
| | | (California, USA); and Malus |
| | | domestica (California, USA). |
| -3-O-[3-hydroxy, 4-methoxy- | 116 | Diospyros dendo (Gabon, Africa) |
| cinnamoyl(trans-)]- | | |
| 2hydroxyursolic acid | | |
| 3-[4-Hydroxycinnamoyl (cis-)], | 188 | Diospyros dendo (Gabon, Africa) |
| 20-hydroxy-ursolic acid | | |
| 3-[4-hydroxycinnamoyl(trans-)]- | 189 | Diospyros dendo (Gabon, |
| 2-hydroxyursolic acid | | Africa); and Malus domestica (California, |
| | | USA) |
| 3-[4-hydroxycinnamoyl(cis-)]- | 190 | Diospyros dendo (Gabon, Africa) |
| 2-hydroxyursolic acid | | |
| Euscaphic acid | 192 | Brazzeia soyauxii (Gabon, Africa) |
| 20B-hydroxyursolic acid | 195 | Arctostaphylos tomentosa |
| | | (California, USA); and |
| | | Arctostaphylos edmundsii |
| | | (California, USA); |
| Tormentic acid | 203 | Brazzeia soyauxii (Gabon, |
| | | Africa) |
| Asiatic acid | 255 | Centella asiatica (Florida, USA) |
| Madecassic acid | 314 | Centella asiatica (Florida, USA) |
| Pygenic acid A | 456 | Pygeum africanum |
| Pygenic acid B | 457 | Pygeum africanum |
| 3-hydroxy-12,20(30) | 430 | Hyptis emoryi (California, USA) |
| ursadienoic acid | | |
| Oleanolic acid | 225 | Vitis L. spp.; Crataegus L. spp. |
| Caulophyllogenin | 323 | Caulophyllum spp. |
| Echinocystic acid | 455 | Albizia spp. |
| 3-acetyl oleanolic acid | 480 | Drosera intermedia |

A general method for isolating the pentacyclic acids triterpenes of the invention can be summarized as follows. First, the plant or plant cell derived material is extracted in an organic solvent, such as ethanol: ethanol acetate (50 EtOH: 50 EtOac). Next, the extract is adsorbing on a powder such as silica and subjected to a series of flash chromatographic separation step gradients. Step gradients of (i) 75% hexanes, 25% ethyl acetate, (ii) 50% hexanes, 50% ethyl acetate, (iii) 100% ethyl acetate, (iv) 75% ethyl acetate, 25% methanol, and (v) 50% ethyl acetate, 50% methanol are typically employed. The 100% ethyl acetate flash chromatography fraction (iii) or FCF3 typically contains the pentacyclic triterpene compound of interest. The FCF3 is then dried and resuspended in a suitable solvent such as methanol: ethyl acetate (70:30) or 100% methanol. Methanol: ethyl acetate (70:30) is a preferred solvent for resuspending FCF3. The dissolved FCF3 fraction is subsequently subjected to preparative high performance liquid chromatography (HPLC). Exemplary HPLC methods entail use of a C18 preparative column that is eluted with 30-70% acetonitrile and fractions are collected every minute. HPLC fractions containing the pentacyclic acid triterpene compound collected, resuspended in a suitable solvent and then identified by a suitable analytical technique such as liquid chromatography electrospray detection mass spectrometry (LC-ELSD-MS). The material contained in the fraction or fractions identified by LC-ELSD-MS or other means is then collected to obtain a pure preparation of the compound.

A variety of illustrative methods that are generally applicable to purifying the pentacyclic acid triterpene compounds of this invention and specifically applicable to purifying certain pentacyclic acid triterpenes are known. Nishimura, et al. (J. Nat. Prod. 1999, 62, 1061-1064) described the identification of 2,3-dihydroxy-24-nor-urs-4(23),12-dien-28-oic acid and 23-hydroxyursolic acid. It is now apparent from the written descriptions contained herein that these compounds will inhibit the formation of biofilms using the procedures described in the examples. Nishimura described procedures to isolate these compounds. Procedures described herein demonstrate these compounds will be contained in flash chromatography fraction 3 (FCF3) as described in the examples. Similar HPLC procedures described herein can be used to further purify these compounds including using a gradient with water with 0.05% TFA and acetonitrile with 0.05% TFA, mobile phase A and B respectively, with a C18 BetaMax Neutral column (250×8 mm; 5 um). The gradient may consist of 40% B isocratic for 5 min, then from approximately 40% to 70% B in 30 min. A skilled artisan would recognize the general applicability of the methods described in Nishimura et al to efficiently isolate either the pentacyclic acid triterpene compounds described herein or structurally related pentacyclic acid triterpenes from various plants and that these compounds will exhibit biofilm inhibition using the procedures described in the examples.

Other illustrative methods that are generally applicable to purifying other pentacyclic acid triterpenes and specifically applicable to purifying certain pentacyclic acid triterpenes are also known. Ballesta-Acosta, et al. (J. Nat. Prod. 2002, 65, p. 1513-1515) described the identification of 2,3-dihydroxy-24-nor-4(23),12-oleanadien-28-oic acid. Begum, et al. (J. Nat. Prod. 1997, 60, p. 20-23) described the isolation of camaldulenic acid also listed at 2,3-dihydroxyolean-11,13(18)-dien-28-oic acid and other related compounds. Chaturvedula, et al. (J. Nat. Prod. 2004, 67, p. 899-901) described the isolation of 3-acetoxy-2-hydroxy ursolic acid, 3-(p-coumaroyl)ursolic acid, and 2,3-diacetoxyursolic acid. Adnyana, et al. (J. Nat. Prod. 2001, 64, p. 360-363) described the isolation of 2,3,6,19-tetrahydroxyoleanolic acid, 2,3,19-trihydroxyoleanolic acid, 2,3,19,23-tetrahydroxyursolic acid, and 2,3,23-trihydroxyoleanolic acid. Ikuta, et al. (J. Nat. Prod. 2003, 66, p. 1051-1054) described the isolation of 2,3-dihydroxyurs-12-en-11-on-28-oic acid and 2,3-dihydroxy-11-methoxyurs-12-en-28-oic acid. Procedures described herein demonstrate these compounds will be contained in FCF3. Similar HPLC procedures described herein can be used to further purify these compounds including using a gradient with water with 0.05% TFA and acetonitrile with 0.05% TFA, mobile phase A and B respectively, with a C18 BetaMax Neutral column (250×8 mm; 5 um). The gradient may consist of 40% B isocratic for 5 min, then from approximately 40% to 70% B in 30 min. A skilled artisan now understands how the written description contained herein can be used to efficiently isolate these compounds and that these compounds will exhibit biofilm inhibition using the procedures described in the examples.

With respect to purifying the compounds of the invention from plant sources, certain embodiments of the invention call for compositions containing more than 1% by weight of a pentacyclic acid triterpene such as ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid and Caulophyllogenin. It is understood that the methods described herein of purifying compounds similar to or identical to the pentacyclic acid triterpenes described in the preceding sentence can be generally employed by those skilled in the art to provide both compositions containing more than 1% by weight of those compounds and processes for making compositions containing more than 1% by weight of those compounds. When stating that these methods can be generally employed, it is further understood that the purification methods described herein also encompass use of similar materials or methods that can be identified through routine experimentation by one skilled in the art. For example, modifications of the HPLC separation conditions identified by routine experimentation may result in improved yield of the desired pentacyclic acid triterpene.

It is likewise contemplated that those embodiments of this invention that call for the presence of one and only one of the pentacyclic acid triterpenes such as ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid and Caulophyllogenin are also enabled by the methods of purification from plant sources described herein. It is understood that the methods described herein of purifying compounds similar to or identical to the pentacyclic acid triterpenes described in the preceding sentence can be generally employed by those skilled in the art to provide both compositions containing one and only of those compounds and processes for making compositions containing one and only of those compounds. When stating that these methods can be generally employed, it is further understood that the purification methods described herein also encompass use of similar materials or methods that can be identified through routine experimentation by one skilled in the art. For example, modifications of the HPLC separation conditions identified by routine experimentation may result in improved separation of the desired pentacyclic acid triterpene from a related pentacyclic acid triterpene present in the plant extract.

Finally, it is further contemplated that those embodiments of this invention that call for an essentially pure preparation of a pentacyclic acid triterpenes such as ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 2β(30) ursadienoic acid, Echinocystic acid, 3-acetyl oleanolic acid and Caulophyllogenin are also enabled by the methods of purification from plant sources described herein. Similarly, it is also understood that those embodiments of this invention that call for compositions containing no or less than 1.0% by weight triterpene glycoside, no or less than 1.0% by weight fatty acid, no or less than 1.0% by weight sterol, or no or less than 1.0% by weight hydrocarbon are also enabled by the methods of purification of pentacyclic acid triterpenes from plant sources described herein. Triterpene glycosides, fatty acids, sterols and hydrocarbons are commonly associated with impure preparations of this invention (Kaufmann, P. in Natural Products from Plants, CRC Press LLC, 1999). As used herein, triterpene glycoside refers to glycosidic derivatives of a triterpene such as asiaticoside, brahmoside, and madecassoside. Fatty acid refer to compounds such as linoleic acid, linolenic acid, and palmitic acid. Sterol refers to compounds such as sitosterol, campesterol and stigmasterol. Hydrocarbons include compounds such as alkanes, alkenes, terpenes, and squalene. Hydrocarbons also include carotenoids such as phytoene, lycopene, or γ-, α-, β-carotene. It is understood that the methods described herein of purifying compounds similar to or identical to the pentacyclic acid triterpenes described in the preceding sentence can be generally employed by those skilled in the art to provide an essentially pure preparation of a pentacyclic acid triterpenes or a preparation of a pentacyclic acid triterpene compound or composition containing no or less than 1.0% by weight triterpene glycoside, no or less than 1.0% by weight fatty acid, no or less than 1.0% by weight sterol, or no or less than 1.0% by weight hydrocarbon. When stating that these methods can be generally employed, it is further understood that the purification methods described herein also encompass use of similar materials or methods that can be identified through routine experimentation by one skilled in the art. For example, modifications of the HPLC separation conditions identified by routine experimentation may result in improved purification of the desired pentacyclic acid triterpene.

It is further anticipated that the compounds of the invention can be obtained by direct synthesis. Direct synthesis may include either total synthesis or semi-synthesis. Both synthetic methods for obtaining these compounds are described below.

Publications illustrate the total synthesis of oleanolic acid and other pentacyclic acid triterpene compounds of the invention. Total synthesis is regarded herein as another means of obtaining the compounds of the invention by direct synthesis. See Corey, E. J. and J. Lee, “Enantioselective Total Synthesis of Oleanolic Acid, Erythrodiol, B-Amyrin, and Other Pentacyclic Triterpenes from a Common Intermediate.” J. Am. Chem. Soc. 1993, 115; 8873-8874. See Huang, A., et al., “An exceptionally short and simple enantioselective total synthesis of pentacyclic triterpenes of the B-Amyrin family.” J. Am. Chem. Soc., 1999, 121; 9999-10003. See Mi, Y., et al., “Total synthesis of (+)-α-onocerin in four steps via four-component coupling and tetracyclization steps.” J. Am. Chem. Soc. 2002, 124; 11290-11291. It is anticipated that the methods taught by these publications will be generally applicable by one skilled in the art to implementing total synthesis of the pentacyclic acid triterpenes of the invention.

Recent publications also illustrate the semi-synthesis of the pentacyclic acid triterpene compounds of the invention. Publications refer to these pentacyclic acid triterpenes as derivatives of ursolic acid and oleanolic acid. These publications also refer to the Carbon number (e.g. C-1 which means Carbon 1) as shown in FIG. 2. This nomenclature will be used within the specification to accurately describe derivatives useful in the practice of this invention. See Farina, C. et al., “Synthesis and anti-ulcer activity of new derivatives of glycyrrhetic oleanolic, and ursolic acids.” II Farmaco. 1998, 53, 22-32. See Honda, T.; et al., “Design and synthesis of 2-Cyano-3,12-Dioxoolean-1,9-dien-28-oic acid, a novel and highly active inhibitor of nitric oxide production in mouse macrophages.” Bioorg. Medic. Chem. Lett., 1998, 8, 2711-2714. See Konoike, T.; et al., “Synthesis of [2-13C]-Oleanolic acid and [2-13C]-Myricerone.” Tetrahedron. 1999, 55; 14901-14914. These publications demonstrate semi-synthetic modifications to positions at C-3, C-28, and C-30; and positions C-2, C-3, and C-12; and positions at C-1, C-2, C-3, and the A-ring, respectively, of the compounds of the invention.

In the semi-synthetic preparation of pentacyclic acid compounds of the invention, a typical starting basic chemical compound may be ursolic acid, oleanolic acid, corosolic acid, asiatic acid, or madecassic acid. In designing semi-synthetic strategies to prepare analogs of the basic chemical compound, modifications at certain positions of the scaffold of the basic chemical compound prove to be important for modulating biofilm inhibition, while other modifications at positions can improve the bioavailability of the compound. Improvement of the bioavailability of the compound would expand the therapeutic range of the compounds by reducing certain cellular toxicities in the subject.

As demonstrated by the biofilm inhibition of corosolic acid and ursolic acid, a hydroxyl group of corosolic acid at position C-2 increases the biofilm inhibition. Modification of position C-19 of corosolic acid, as exemplified in tormentic acid slightly reduces the biofilm inhibition against P. aeruginosa. However, modification of corosolic acid at positions C-3 (hydroxycinnamoyl) and C-20 (hydroxyl), as exemplified in 3-[4-Hydroxycinnamoyl (cis-)], 20-hydroxy-ursolic acid, increases biofilm inhibition against P. aeruginosa. These examples merely demonstrate that the modifications at certain positions of the ursane or oleanane scaffold can modulate the magnitude of biofilm inhibition. The examples are not meant to limit the scope of claimed invention.

Finally, another source of the pentacyclic acid triterpenes of the invention are commercial sources or vendors. Purified forms of corosolic acid, ursolic acid, oleanolic acid, madecassic acid, asiatic acid, pygenic acid (A or B), caulophyllogenin and echinocystic acid may be obtained from a commercial source. For example, ursolic acid and oleanolic acid may be purchased from Sigma-Aldrich Chemical Company (St. Louis, Mo., USA) and corosolic acid, asiatic acid, madecassic acid, pygenic acid (A or B), caulophyllogenin and echinocystic acid may be purchased from Chromadex (Santa Ana, Calif., USA). The compounds obtained from commercial sources may be furthered separated and purified as needed using methods such as column chromatography, high pressure liquid chromatography (HPLC), and/or recrystallization described herein. As will be appreciated by the skilled artisan, further methods of synthetically producing and derivatizing the compounds disclosed herein will be evident from this specification. Additionally, the various isolation, purification, and/or synthetic steps may be performed in an alternate sequence or order to produce the desired compounds.

Prodrugs of the pentacyclic acid triterpene compounds described herein are also anticipated. As used herein, the term “prodrug” means a derivative of an active compound (drug) that undergoes a transformation under the conditions of use, such as within the body, to release an active drug. It is understood that this application discloses pentacyclic acid triterpenes that are active compounds and that may be converted to prodrugs by the methods described herein. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

A wide variety of progroups, as well as the resultant promoieties, suitable for masking functional groups in active compounds to yield prodrugs are well-known in the art. For example, a hydroxyl functional group may be masked as a sulfonate, ester or carbonate promoiety, which may be hydrolyzed in vitro to provide the hydroxyl group. An amino functional group may be masked as an amide, imine, phosphinyl, phosphonyl, phosphoryl or sulfenyl promoiety, which may be hydrolyzed in vivo to provide the amino group. A carboxyl group may be masked as an ester (including silyl esters and thioesters), amide or hydrazide promoiety, which may be hydrolyzed in vivo to provide the carboxyl group. Other specific examples of suitable progroups and their respective promoieties will be apparent to those of skill in the art.

In the present invention, a “progroup” means a type of protecting group that, when used to mask a functional group within an active drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH3 comprises the progroup —C(O)CH3.

Specific prodrug embodiments of the compounds of the invention include derivatives of the C-3 hydroxyl group and/or the C-28 carboxyl group of the ursane and oleanane scaffolds that represent pentacyclic acid triterpene compounds of the invention (FIG. 2). One set of prodrugs contemplated by this invention are esters, sulfonates, and carbonates of the C-3 hydroxyl group of a compound of the invention. Another set of prodrugs contemplated by this invention include esters, amides, and hydrazides of the C-28 carboxyl group of a compound of the invention.

Pharmaceutical Compositions

Pharmaceutical compositions comprising an antimicrobial agent, a compound selected from the group consisting of ursolic acid, oleanolic acid, 30-hydroxyursolic acid, 2-hydroxyoleanolic acid, Corosolic acid, 3-O-[3-hydroxy, 4-methoxycinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 2β(30) ursadienoic acid, Echinocystic acid, 3-acetyl oleanolic acid, Caulophyllogenin and salts, hydrates, solvates, prodrugs and N-oxides thereof, and a pharmaceutically acceptable carrier are contemplated by this invention. Preferred pentacyclic acid triterpene compounds used in the compositions of the instant invention include 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, and madecassic acid, as well as salts, hydrates, solvates, prodrugs and N-oxides thereof. More preferred pentacyclic acid triterpene compounds used in the compositions of the instant invention include corosolic acid, asiatic acid, and madecassic acid, as well as salts, hydrates, solvates, prodrugs and N-oxides thereof. The most preferred pentacyclic acid triterpene compound used in the compositions of the instant invention is corosolic acid or a salt, hydrate, solvate, prodrug or N-oxides thereof. It is anticipated that combining the compounds of this invention with an antimicrobial agent and a pharmaceutically acceptable carrier will yield a superior pharmaceutical composition for either preventing, inhibiting or reducing a biofilm or its formation or for treating, controlling, reducing or preventing a bacterial infection in a subject in need thereof. The antimicrobial agent may be selected from the group consisting of triclosan, metronidazole, tetracyclines, quinolones, plant essential oils, camphor, thymol, carvacrol, menthol, eucalyptol, methyl salicylate, tobramycin, clindamycin, ciprofloxacin, rifampin, oxfloxacin, macrolides, penicillins, cephalosporins, amoxicillin/clavulanate, quinupristin/dalfopristin, amoxicillin/sulbactum, fluoroquinolones, ketolides, and aminoglycosides. For dentrifices, it is envisioned that the antimicrobial agent is selected from a preferred group consisting of consisting of triclosan, metronidazole, tetracyclines, quinolones, plant essential oils, camphor, thymol, carvacrol, menthol, eucalyptol, and methyl salicylate. For other compositions useful as oral, topical, parenterally injected, percutaneous, rectal, intranasal or inhaled dose forms it is envisioned that the antimicrobial agent is an antibiotic selected from the group consisting of tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, and aminoglycosides.

In one embodiment of this invention, a compound of the invention selected from the group consisting of pentacyclic acid triterpenes such as ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof is present at more than 1% by weight. In certain embodiments, the pentacyclic acid triterpene compound of the invention comprises 2% to about 60% by weight of the composition. In particular, it anticipated that oral dose forms of the composition may comprise over 30% by weight of the pentacyclic acid triterpene compound. In certain preferred embodiments useful as topical treatments or dentifrices, the pentacyclic acid triterpene compound makes up about 2% to about 5% by weight of the composition. In the most preferred embodiments useful as topical treatments or dentifrices, the pentacyclic acid triterpene compound makes up about 2% by weight of the composition.

In other embodiments of the invention, the composition comprises an antimicrobial agent, one and only one compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof, and a pharmaceutically acceptable carrier, and wherein the compound is present in a concentration of at least about 0.1% by weight, based on the total weight of the composition. While not being limited by theory, it is believed that in certain instances compositions that provide one and only one pentacyclic acid triterpene compound may provide improved control of biofilms or bacterial infections in subjects in need thereof. In particular, it anticipated that oral dose forms of the composition may comprise over 30% by weight of one and only one the pentacyclic acid triterpene compound. In certain preferred embodiments useful as topical treatments or dentifrices, one and only one pentacyclic acid triterpene compound makes up about 2% to about 5% by weight of the composition. In the most preferred embodiments useful as topical treatments or dentifrices, one and only one pentacyclic acid triterpene compound makes up about 2% by weight of the composition.

In other embodiments of the invention, the composition comprises an antimicrobial agent, at least 0.1% by weight a compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20 β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof and a pharmaceutically acceptable carrier, wherein said pharmaceutical composition contains no or less than 1.0% by weight triterpene glycoside, no or less than 1.0% by weight fatty acid, no or less than 1.0% by weight sterol, or no or less than 1.0% by weight hydrocarbon. Without being limited by theory, it is believed that in certain instances compositions that contain no or less than 1% by weight triterpene glycoside, no or less than 1.0% by weight fatty acid, no or less than 1.0% by weight sterol, or no or less than 1.0% by weight hydrocarbon will provide improved efficacy (i.e. control of biofilm formation or control of bacterial infection) and less prone to causing undesirable side effects in subjects. In particular, it anticipated that oral dose forms of the composition may comprise over 30% by weight of one and only one the pentacyclic acid triterpene compound. In certain preferred embodiments useful as topical treatments or dentifrices, one and only one pentacyclic acid triterpene compound makes up about 2% to about 5% by weight of the composition. In the most preferred embodiments useful as topical treatments or dentifrices, one and only one pentacyclic acid triterpene compound makes up about 2% by weight of the composition.

Various pharmaceutical compositions that may be used in the present invention, including the compounds of the invention and the specific examples described herein, further including pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a patient, is capable of providing (directly or indirectly) a compound used in this invention. The compositions useful in the present invention may, optionally, be converted to their therapeutically-active non-toxic acid salt forms by treatment with appropriate acids. Such acids include inorganic acids, e.g., hydrochloric and hydrobromic acids, sulfuric acid, nitric acid, phosphoric acid and like acids; or organic acids, such as acetic, propanoic, hydroxyacetic, 2-hydroxypropanoic, 2-oxo-propanoic, ethanedioic, propanedioic and like acids. Of course, the salt forms may be converted into the free base form by treatment with alkali. The pharmaceutically-acceptable acid salts of the present invention also comprise the solvates that the compositions of the present invention may form, which, of course, are included within the scope of the present invention. Non-limiting examples of such solvates are hydrates, alcoholates and the like.

Such pharmacologic compositions may be formulated in various ways known in the art for administration purposes. To prepare the pharmaceutical compositions of the present invention, an effective amount of the particular compound, in base or acid salt form, as the active ingredient is combined with one or more pharmaceutically-acceptable carriers and delivery vehicles. Numerous pharmaceutically acceptable carriers and delivery vehicles exist that are readily accessible and well-known in the art, which may be employed to generate the preparation desired (i.e. that permit administration of the pharmaceutical composition orally, topically, rectally, percutaneously, by parenteral injection, intranasally or by inhalation). Representative examples of pharmaceutically acceptable carriers and delivery vehicles include aluminum stearate, lecithin, serum proteins, such as human serum albumin; buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based substances, polyethylene glycol, sodium carboxymethylcellulose, polyarylates, waxes, polyethylene, polyoxypropylene-block polymers, polyethylene glycol and wool fat, and the like.

The pharmacologic compositions described herein may further be prepared in unitary dosage form suitable for administration orally, percutaneously, by parenteral injection (including subcutaneous, intramuscular, intravenous and intradermal), topically, intranasally, by inhalation, or for application to a medical device, such as an implant, catheter, or other device. In preparing the compositions that permit administration of an oral dosage, for example, any of the pharmaceutically acceptable carriers known in the art may be used, such as water, glycols, oils, alcohols and the like in the case of carriers that permit oral delivery of liquid preparations such as suspensions, syrups, elixirs and solutions. When solid pharmaceutically acceptable carriers are desired that permit oral or rectal administration, starches, sugars, kaolin, lubricants, binders, cellulose and its derivatives, and disintegrating agents and the like may be used to prepare, for example, powders, pills, capsules and tablets.

For pharmaceutically acceptable carriers that permit parenteral administration, the pharmaceutically acceptable carriers often comprise sterile water, which may be supplemented with various solutes to, for example, increase solubility. Injectable solutions may be prepared in which the pharmaceutically acceptable carrier comprises saline solution, glucose solution, or a mixture thereof, which may include certain well-known anti-oxidants, buffers, bacteriostats, and other solutes that render the formulation isotonic with the blood of the intended patient.

For pharmaceutically acceptable carriers that permit intranasal administration, the pharmaceutically acceptable carriers often comprise poly acrylic acids such as Carbopol® 940, a hydrogenated castor oil such as Cremophor® RH40, glycerol, vinylpyrrolidones such as PVP-K90® or PVP K30®, polyethylene glycols such as PEG 1450®, benzyl alcohol, Edetate sodium, hydroxycellulose, potassium chloride, potassium phosphate, and sodium phosphate. Compositions used for intranasal administration also commonly include benzalkonium chloride as an anti-microbial preservative.

For pharmaceutically acceptable carriers that permit administration by inhalation, the pharmaceutically acceptable carriers often comprise solvent/carrier/water mixtures that are easily dispersed and inhaled via a nebulizer or inhaler. For example, a mixture of ethanol/propylene glycol/water in the ratio of about 85:10:5 (parts ethanol: parts propylene glycol: parts water) can be used to administer the compounds and compositions of the invention via inhalation.

For pharmaceutically acceptable carriers that permit percutaneous administration, the pharmaceutically acceptable carrier may, optionally, comprise a penetration enhancing agent and/or a suitable wetting agent.

Dosage forms that permit topical or transdermal administration of a compound of this invention include ointments, pastes, creams, lotions, gels, powders, solutions, sprays, inhalants or patches. The active compound or compounds is/are mixed under sterile conditions with a pharmaceutically acceptable carrier and optionally one or more preservatives and/or buffers. In the context of certain embodiments of this invention, the active compound is a pentacyclic acid triterpene. In the context of other embodiments of this invention, the pentacyclic acid triterpene is combined in the composition with another active compound that is an antimicrobial agent or antibiotic.

The ointments, pastes, creams and gels may contain, in addition to an active compound or compounds according to the present invention, pharmaceutically acceptable carriers that permit topical or transdermal administration such as animal and vegetable fats, oils, waxes, paraffins, starch, tragacanth, cellulose derivatives, polyethylene glycols, silicones, bentonites, silicic acid, talc and zinc oxide, or mixtures thereof.

In some cases, the pH of the pharmaceutical formulations contemplated herein may be adjusted with acceptable acids, bases or buffers to enhance the stability of one or more of the active compounds present or their delivery forms. In the context of certain embodiments of this invention, the active compound is a pentacyclic acid triterpene. In the context of other embodiments of this invention, the pentacyclic acid triterpene is combined in the composition with another active compound that is an antimicrobial agent or antibiotic.

Still further, in order to prolong the anti-bacterial effect of a compound disclosed herein, it may be desirable to slow the absorption of the compound from subcutaneous or intramuscular injection. This may be accomplished using a liquid suspension of crystalline or amorphous material with poor water solubility. The rate of absorption of the compound then depends upon its rate of dissolution, which, in turn, may depend upon crystal size and crystalline form. Alternatively, delayed absorption of a parenterally administered drug form may be accomplished by dissolving or suspending the compound in an oil vehicle.

Injectable depot forms are made, e.g., by forming microencapsule matrices of one or more compounds of the present invention in biodegradable polymers such as polylactide-polyglycolide. Depending upon the ratio of active(s) to polymer and the nature of the particular polymer employed, the rate at which such active(s) is released may be controlled. Examples of other biodegradable polymers include poly(orthoesters) and poly(anhydrides). Depot injectable formulations are also prepared by entrapping the compound in liposomes or microemulsions that are compatible with body tissues.

The pharmaceutical composition is may also be a dentifrice. In the present invention, “dentifrice” is understood to broadly include compositions suitable for administering to the oral cavity, especially, for example, to the gingival/mucosal tissue or to the teeth. Thus, the dentifrice may include toothpastes, toothpowders, liquid dentifrices, mouth detergents, mouthwashes, troches, chewing gums, dental or gingival massage creams, dental strips, dental gels, and gargle tablets.

When the pharmaceutical composition of this invention is a dentifrice such as tooth paste, a tooth or gum adherence promoting substance selected from the group consisting of copolymers of methyl vinyl ether and maleic anhydride, copolymers of vinyl pyrrolidone and vinyl acetate, and cyclodextrins may also be included in the composition. Copolymers of methyl vinyl ether and maleic anhydride useful in this invention may have molecular weights ranging from 200,000 to 2,000,000 kD and may be free acids, mixed sodium and calcium salts, or half ester derivatives. Representative commercial sources of the copolymers of methyl vinyl ether and maleic anhydride include GANTREZ® AN (CAS # 9011-16-9) GANTREZ® S (CAS # 25153-40-69) GANTREZ® MS (CAS# 62386-95-2) GANTREZ® ES (CAS# 25087-06-3 or CAS# 25119-68-0) and can be obtained from International Specialty Products Wayne, New Jersey. Copolymers of vinyl pyrrolidone and vinyl acetate useful in the invention typically have a molecule weight of approximately 27,000 kD and are water soluble. Representative commercial sources of the copolymers of vinyl pyrrolidone and vinyl acetate PLASDONE® S-630 and can be obtained from International Specialty Products Wayne, N.J. Cyclodextrins useful in the invention are cyclic oligosaccharides composed of either 6, 7 or 8 glucose units (a-, b- and g-cyclodextrin, respectively). Representative commercial sources of the cyclodextrins useful in this invention include CAVAMAX®W6 Pharma, CAVAMAX®W7 Pharma and CAVAMAX®W8 Pharma (a-, b- and g-cyclodextrin, respectively) and can be obtained from International Specialty Products Wayne, N.J.

When the composition of this invention is a dentifrice, an antimicrobial agent is selected from the group consisting of triclosan, metronidazole, tetracyclines, quinolones, plant essential oils, camphor, thymol, carvacrol, menthol, eucalyptol, and methyl salicylate may also be included. Pharmaceutically acceptable carriers that permit administration of the pentacyclic acid triterpene compounds of this application as dentifrices include sorbitol, glycerin, silica, sodium lauryl sulfate and Xanthum gum. The dentifrices of this invention may also include sodium fluoride.

Methods of Inhibiting Biofilm Formation

Various methods for inhibiting biofilm formation both in vivo and in vitro are contemplated by this invention. In these methods, either a composition containing a pentacyclic acid triterpene compound or the pentacyclic acid triterpene compound itself may be provided to the system before, during, or after a biofilm has formed. The pentacyclic acid triterpene compound used in the biofilm inhibition methods is selected from the group consisting of ursolic acid, oleanolic acid, 30-hydroxyursolic acid, 2-hydroxyoleanolic acid, Corosolic acid, 3-O-[3-hydroxy, 4-methoxycinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof. Preferred pentacyclic acid triterpene compounds used in the methods of the instant invention include 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, and madecassic acid, as well as salts, hydrates, solvates, prodrugs and N-oxides thereof. More preferred pentacyclic acid triterpene compounds used in the methods of the instant invention include corosolic acid, asiatic acid, and madecassic acid, as well as salts, hydrates, solvates, prodrugs and N-oxides thereof. The most preferred pentacyclic acid triterpene compound used in the methods of the instant invention is corosolic acid or a salt, hydrate, solvate, prodrug or N-oxides thereof.

In the methods for inhibiting biofilms, an antimicrobial agent, antibiotic or biocide may be incorporated into the system together with the compound in a composition or administered separately. In the present invention, any antimicrobial agent, antibiotic or biocide may be used. Representative examples of biocides that may be used in the present invention, include isothiazolone, derivatives thereof, compounds having a isothiazolone functions, 3-isothiazolone compound, 5-chloro-2-methyl-3-isothiazolone, 1-methyl-3,5,7-triaza-1-azoniatricyclo (3.3.1.1) deoane chloride, 4,5-dichloro-2-octyl-3 isothiazolone, 2-bromo-2-nitropropanediol, 5-bromo-5-nitro dioxane, thiocyanomethylthiobenzothiazole, 4,5-dichloro-2-octyl-3-isothiazolone and 2-noctyl-3-isothiazolone, tetrachloroisophalonitrile, 1,2-benzisothiazolin-3-one, 2-methyl-4,5-trimethylene-4-isothiazolin-3-one, 5-chloro-2-methyl-4 isothiazolin-3-one, 2-methyl-4-isothiazolin-3-one, 4-(2-nitrobutyl)morpholine, beta-nitrostyrene (“NS”), beta-bromobeta-nitrostyrene (“BNS”), methylehloro/isothiazolone (“IZN”), methylenebisthiocyanate(“MBT”), 2,2dibrortmo-3-nitrilopropionamide (“DBNPA”), 2-bromo-2-bromomethylglutaronitrile(“BBMGN”), alkyldimethylbenzylammonium chloride (“ADBAC”), and betatiitrovinylfuran (“NVF”), 2-methyl-3-isothiazolone, methylene bisthiocyanate, ptolyldiiodotnethylsulfone, 2-methylthio-4-tertbutylamino-6-cyclopropylamino-s-tiiazine,N,N-dimethyl-N′phenyl(N′fluorodiehloromethylthio)sulfainide, sulfamides, N-(cyclo)alkyl-isothiazolone, benzisothiazolin-3-one, etc. and their mixtures.

Other examples of biocides that may be combined with one or more of the biocides listed above include bicyclic oxazolidoines and their mixtures, amine-basedbactericide, polyacrolein copolymer, 4,4-dinethyloxazolidine, 2((hydroxymethyl)-amino)ethanol, mixtures of 1,2-benzisothiazolone-3-one with one or more amines, tetrahydro-3,5-dimethyl-2H-1,3,5-thiadiazitie-2-thione. 1.2-benzisothiazolin-3-one,tetrachloroisophthalonitrile, N-cyclopropyl-N-(1,1-dimethylethyl)-6-(methylthio)-1,3; 5-triazine-2,4-diamine, mixtures of N-cyclopropyl-N-(1,1-dimethylethyl)-6-(methylthio)-1,3,5-triazine-2,4-diamine with tetrachloroisophthalonitrile, mixtures of tetrachloroisophthalonitrile with 3-iodo-2-propynylbutyl carbamate, N-(trichloromethylthio)-phthalimide, 3iodo-2-propynylbutyl carbamate, tetrachloroisophthalonitrile, and their mixtures.

Representative examples of antimicrobial agents useful in methods for inhibiting biofilms include triclosan, metronidazole, tetracyclines, quinolones, plant essential oils, camphor, thymol, carvacrol, menthol, eucalyptol, methyl salicylate, tobramycin, clindamycin, ciprofloxacin, rifampin, oxfloxacin, macrolides, penicillins, cephalosporins, amoxicillin/clavulanate, quinupristin/dalfopristin, amoxicillin/sulbactum, fluoroquinolones, ketolides, and aminoglycosides.

Representative examples of antibiotics that may be useful in the practice of the methods of this invention include tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, or aminoglycosides.

In this application of this method, the compound may be applied to the surface of a substrate. The substrate may be made from any material to which the compound or a composition containing the compound may be applied. Representative examples of the kinds of materials from which the substrate may be made, include porous materials, soft materials, hard materials, semi-hard materials, regenerating materials, and non-regenerating materials. Preferably, the substrate is made from an inert material selected from the group consisting of a polymer, a metal, an alloy, and combinations thereof. In an alternatively preferred embodiment, the substrate is a biological structure, such as for example, regenerating proteins of mammalian cellular membranes, dental enamel, gum, tongue, and biological polymers.

Preferably, the substrate is a surface of a device that is susceptible to biofilm formation. Examples of suitable substrate surfaces according to the present invention include vessel hulls, automobile surfaces, air plane surfaces, membranes, filters, industrial machinery, microtiter plates, continuous flow chambers, bioreactors, fermentors, chemostats and industrial equipment. Bioreactors can be purchased from Biosurface Technologies Corporation (Bozeman, Mont., USA) and are preferably a drip flow reactor and more preferably a Centers for Disease Control reactor (CDC reactor) or a Rotating Disk Reactor (RDR).

The substrate may also be a medical device. Examples of medical devices included within the present invention include any device that is capable of being implanted temporarily or permanently into a mammalian organism, such as a human. Representative examples of medical devices that may be used according to the present invention include: central venous catheters, urinary catheters, endotracheal tubes, mechanical heart valves, pacemakers, vascular grafts, stents, and prosthetic joints.

Methods of Preventing or Inhibiting Bacterial Infections in a Subject

The methods of the present invention include using the compositions described herein to prevent or inhibit bacterial infections. In the case of medical applications where the subject is a human, the methods of the present invention comprise the steps of providing an effective amount of at least one composition described herein to a patient. In the case of veterinary applications, the subject is an animal. Such compositions and methods are used to treat and/or prevent bacterial-related health afflictions either alone or in combination with antimicrobial agent. In the methods for preventing or inhibiting bacterial infections, an antimicrobial agent, antibiotic or biocide may be incorporated into the system together with the pentacyclic acid triterpene compound in a composition or administered separately. Representative examples of antimicrobial agents that may be useful in the practice of this invention include triclosan, metronidazole, tetracyclines, quinolones, plant essential oils, camphor, thymol, carvacrol, menthol, eucalyptol, methyl salicylate, tobramycin, clindamycin, ciprofloxacin, rifampin, oxfloxacin, macrolides, penicillins, cephalosporins, amoxicillin/clavulanate, quinupristin/dalfopristin, amoxicillin/sulbactum, fluoroquinolones, ketolides, and aminoglycosides. The antimicrobial agent may be an antibiotic. Representative examples of antibiotics that may be useful in the practice of this invention include tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, or aminoglycosides. While the following description makes reference to specific methods and uses of the disclosed compositions for human applications, it should be appreciated that such compositions and methods may be equally useful in veterinary applications wherein the subject is an animal.

Asiatic acid and madecassic acid are shown herein to prevent, reduce and/or inhibit biofilm formation by P. aeruginosa and E. coli in the absence of any bacterial growth inhibition. The examples shown herein further demonstrate that asiatic acid and madecassic acid can be used to treat chronic infections involving biofilms, including urinary tract infection, gastritis, lung infection, ear infection, cystitis, pyelonephritis, arterial damage, leprosy, tuberculosis, benign prostatic hyperplasia, prostatitis, osteomyelitis, bloodstream infection, cirrhosis, skin infection, acne, rosacea, open wound infection, chronic wound infection, and sinus infection. Other compounds in Centella asiatica extract unnecessarily dilute the concentration of asiatic acid or madecassic acid and reduce their efficiency. We demonstrate that asiatic acid and madecassic acid are biofilm inhibitors and can be used to control, prevent, or treat bacterial infections involving biofilms like urinary tract infections, cystitis, pyelonephritis, and ear infections with or without antibiotics. The use of asiatic acid or madecassic acid as anti-infectives was not previously contemplated as these compounds do not display direct anti-bacterial activity when assayed in bacterial growth inhibition studies.

According to the methods of preventing or inhibiting bacterial infections of animals disclosed herein, the bacterial infections are treated or prevented in a patient by administering or providing an effective amount of a pentacyclic acid triterpene compound or composition disclosed herein, in such amounts and for such time as is necessary to achieve the desired result. The pentacyclic acid triterpene compound used in the methods is selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof. Preferred pentacyclic acid triterpene compounds used in the methods of the instant invention include 30-hydroxyursolic acid, 2-hydroxyoleanolic acid, corosolic acid, 3-O-[3-hydroxy, 4-methoxycinnamoyl(trans-)]-2-hydroxyursolic acid, asiatic acid, and madecassic acid, as well as salts, hydrates, solvates, prodrugs and N-oxides thereof. More preferred pentacyclic acid triterpene compounds used in the methods of the instant invention include corosolic acid, asiatic acid, and madecassic acid, as well as salts, hydrates, solvates, prodrugs and N-oxides thereof. The most preferred pentacyclic acid triterpene compound used in the methods of the instant invention is corosolic acid or a salt, hydrate, solvate, prodrug or N-oxides thereof.

The specific therapeutically effective dose level for any particular patient may depend upon a variety of factors, including the specific biofilm (and, preferably, taking into account the source of such biofilm) being treated or inhibited; the amount of existing biofilm to be treated, if any, within a given patient; the activity of the specific compound employed; the specific pharmacologic formulation employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts. Furthermore, it may be appropriate to administer the required dose more than once in a twenty-four hour period, such as for example in two, three, four or more sub-doses at appropriate intervals throughout the day.

By way of example only, the total daily dose of one or more of the biofilm inhibitors disclosed herein may be provided to a patient in single or in divided doses, which may be in amounts from 0.01 to 50 mg/kg body weight or, more typically, from 0.1 to 25 mg/kg body weight. Single dose compositions may contain such amounts or submultiples thereof to make up the daily dose. More preferably, treatment regimens according to the present invention may comprise administering to a patient about 10 mg to about 1000 mg of the biofilm inhibitor(s) disclosed herein, per day in single or multiple doses.

More than 1 million patients develop urinary tract infections from catheters. The present invention may be utilized to inhibit biofilms in or on urinary catheters and, further, to reduce or prevent bacterial colonization thereon. The compounds and compositions of the present invention also may be used to inhibit biofilms formed by E. coli that reside intracellularly in bladder cells, which resist conventional antibiotics and evade host immune systems. Not wishing to be bound by a particular theory, it is believed that by preventing or disrupting the attachment of E. coli to uroplakin or the proteins of the tight junctions of umbrella cells of the bladder, the compounds and compositions of the present invention may prevent, reduce, or control the re-occurrence of such urinary tract infections.

The compounds and compositions of the present invention also may be used to treat, i.e., prevent and/or reduce the risk of atherosclerosis and kidney stones. Again, not wishing to be bound by a particular theory, it is believed that bacterial colonization may cause atherosclerosis and the formation of kidney stones. For example, bacterial colonization has been identified in calcified human aneurysms, carotid plaques, femoral arterial plaques, and cardiac valves. Arterial calcification appears to resemble infectious lesion formation in models of atherosclerosis. Moreover, it is believed that a toxin produced by Cag-A positive Helicobacter pylori colonization of the stomach leads to tissue inflammation and lesions in arterial walls resulting in atherosclerosis. Accordingly, administering to a patient in need thereof one or more compounds of the present invention (or a composition containing one or more compounds of the present invention) may reduce the risk of, or treat atherosclerosis and kidney stones.

The compounds and compositions of the present invention may be used to treat cystic fibrosis. The principal organism found in the lungs of cystic fibrosis patients is Pseudomonas aeroginosa, existing within a biofilm. Thus, the compounds and compositions of the present invention may be used to prevent, inhibit or reduce the formation of biofilms in the lungs of such cystic fibrosis patients.

Diseased tissue, including certain tumors, are more susceptible to bacterial colonization. Based on this observation, clostridia spores and attenuated Salmonella typhimurium have been used to deliver therapeutic proteins to tumors. These bacteria selectively colonize tumors versus normal tissue. Accordingly, further embodiments of the invention include administering the compounds and compositions of the present invention to diseased tissues to reduce, treat or eradicate the biofilms within the diseased tissue, including tumors. Again, not wishing to be bound by a particular theory, it is believed that the eradication of biofilms and bacteria from such diseased tissue would enable the mammalian immune system, and/or other pharmaceutical compositions, to further treat the diseased tissue after bacterial colonization has been removed or reduced.

The compounds and compositions of the present invention may also be administered to patients experiencing gastritis. While not wishing to be bound by a particular theory, it is believed that the compounds and compositions of the present invention may be used to prevent the attachment of Helicobactor pylori to gastric epithelial cells, which retards the bacteria's ability to invade these cells and/or inhibits or reduces subsequent virulence factors that result in inflammation. By preventing H pylori attachment to gastric epithelial cells, such biofilm inhibitors may further mitigate arterial damage, which may otherwise lead to an increased risk of stroke.

Notwithstanding the examples set forth above, those skilled in the art should appreciate that the compounds and compositions of the present invention may generally be employed to reduce, cure, and/or prevent other acute or chronic microbial infections caused by, e.g., bacterial colonization not expressly described herein. Such compounds and compositions may be used to control, for example, microorganisms that colonize extracellularly or intracellularly. By way of further illustration only, such compounds and compositions may be used to reduce, cure and/or prevent: arterial damage, gastritis, urinary tract infections, otitis media, leprosy, tuberculosis, benign prostratic hyperplasia, chronic prostratitis, chronic infections of humans with cystic fibrosis, osteomyelitis, bloodstream infections, skin infections, open wound infections, and any acute or chronic infection that involves a biofilm.

As previously stated in this specification, conserved mechanisms of bacterial pathogenesis among Gram-positive and Gram-negative bacteria involve cellular invasion. This process enables the bacteria to evade an immune response to increase their population. Therefore, compounds that reduce bacterial invasion would significantly assist the immune system in the eradication of these pathogens. A reduction in bacterial invasion into cells would also help increase the effectiveness of conventional antibiotics. Niels Moller-Frimodt described that antibiotics used to treat urinary tract infections efficiently kill bacteria in the urine, but are insufficient to kill bacteria after they invade the bladder or tissues (Moller-Frimodt, N. Int. J. of Antimicrob Agents, 2002, 19; 546-553). This further supports the benefits of compounds that reduce the invasion of bacteria into cells.

Preventing or Inhibiting Bacterial Infections of Plants

Finally, bacterial infections may also be prevented or inhibited by the compositions containing pentacyclic acid triterpene compounds disclosed herein when the subject is a plant. Thus, the compound or a composition containing the pentacyclic acid triterpene compound may be administered to a plant, such as a surface of a plant to prevent or inhibit the formation of a biofilm on the plant. Methods employing compositions comprising an antimicrobial agent, a compound selected from the group consisting of ursolic acid, oleanolic acid, 30-hydroxyursolic acid, 2-hydroxyoleanolic acid, Corosolic acid, 3-O-[3-hydroxy, 4-methoxycinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof, and a agriculturally acceptable carrier are contemplated by this invention. Preferred pentacyclic acid triterpene compounds used in the methods of the instant invention include 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, and madecassic acid, as well as salts, hydrates, solvates, prodrugs and N-oxides thereof. More preferred pentacyclic acid triterpene compounds used in the methods of the instant invention include corosolic acid, asiatic acid, and madecassic acid, as well as salts, hydrates, solvates, prodrugs and N-oxides thereof. The most preferred pentacyclic acid triterpene compound used in the methods of the instant invention is corosolic acid or a salt, hydrate, solvate, prodrug or N-oxides thereof. Representative types of plants to which the compound or composition of the present invention may be applied include, for example, corn, maize, soybean, wheat, rice, and canola plants. The compound or composition may also be applied to vegetable and fruit crops prone to bacterial disease such as apples, apricots, cherries, nectarines, peaches, pears, plums, prunes, quince almonds, chestnuts, filberts, pecans, pistachios, walnuts, citrus, blackberries, blueberries, boysenberries, cranberries, currants, loganberries, raspberries, strawberries, grapes, avocados, bananas, kiwi, persimmons, pomegranate, pineapple, tropical fruits, artichokes, kohlrabi, arugula, leeks, asparagus, lentils, beans, lettuce (e.g., head, leaf, romaine), beets, bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, peas, chinese cabbage, peppers, collards, potatoes, cucumber, pumpkins, cucurbits, radishes, dry bulb onions, rutabaga, eggplant, salsify, escarole, shallots, endive, soybean, garlic, spinach, green onions, squash, greens, sugar beets, sweet potatoes, turnip, swiss chard, horseradish, tomatoes, kale, and turnips.

It is anticipated that the methods described herein will be applicable to preventing or inhibiting a variety of bacterial infections of plants. Pseudomonadaceae, Rhizobiaceae, Enterobacteriaceae, Corynebacteriaceae and Streptomycetaceae bacteria are all economically significant plant pathogens that may be controlled by the present invention. Non-limiting examples of specific plant pathogens that may be effectively inhibited by the methods described herein include: Xanthomonas species, such as, for example, Xanthomonas campestris pv. oryzae; Pseudomonas species, such as, for example, Pseudomonas syringae pv. lachrymans; and Erwinia species, such as, for example, Erwinia amylovora. It is also anticipated that the methods of preventing or inhibiting bacterial infections of plants described herein may also include use of compositions further comprised of antimicrobial agents such as bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Methods of preventing or inhibiting bacterial infections described herein can be used to treat all plants and parts of plants. By plants are understood here all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties which can or cannot be protected by varietal property rights. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment of the plants and the parts of plants with the active compounds according to the invention is carried out directly or by action on their surroundings, habitat or storage space, according to customary treatment methods, for example by dipping, spraying, evaporating, atomizing, broadcasting, spreading-on and, in the case of propagation material, in particular in the case of seeds, furthermore by one- or multi-layer coating. In the context of certain embodiments of this invention, the active compound is a pentacyclic acid triterpene. In the context of other embodiments of this invention, the pentacyclic acid triterpene is combined in the composition with another active compound that is an antimicrobial agent or antibiotic.

Agriculturally Acceptable Carriers and Compositions

Depending on their particular physical and/or chemical properties, the pentacyclic acid triterpene compounds and compositions can be converted to the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the pentacyclic acid triterpene compounds and compositions with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The pentacyclic acid triterpene compounds and compositions can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants. In the context of certain embodiments of this invention, the active compound is a pentacyclic acid triterpene. In the context of other embodiments of this invention, the pentacyclic acid triterpene is combined in the composition with another active compound that is an antimicrobial agent or antibiotic.

The pentacyclic acid triterpene compounds and compositions according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

### EXAMPLES

The following examples illustrate the use of compounds of the present invention and the preparation of formulations comprising these compounds. The examples demonstrate many uses of the compounds and are not intended to limit the scope of the present invention. Those of skill in the art should, in light of the present disclosure, appreciate that many changes can be made in the specific embodiments which are disclosed and still obtain a like or similar result without departing from the spirit and scope of the invention.

### Example 1

Inhibition of Biofilm Formation by Asiatic acid, Corosolic acid, and Madecassic acid: Escherichia coli clinical strain UT189 and laboratory strain JM109 Assays.

Biofilm inhibition experiments were conducted using an assay adapted from the reported protocol described in Pratt and Kolter, 1998, Molecular Microbiology, 30: 285-293; Li et al., 2001, J. Bacteriol., 183: 897-908. E. coli clinical strain UT189 was grown in LB in 96 well plates at room temperature for one or two days without shaking. E. coli laboratory strain JM109 was grown in LB plus 0.2% glucose in 96 well plates at room temperature for one day without shaking. To quantify the biofilm mass, the suspension culture was poured out and the biofilm was washed three times with water. The biofilm was stained with 0.1% crystal violet for 20 minutes. The plates were then washed three times with water. OD reading at 540 nm was measured to quantify the biofilm mass at the bottom of the wells. Then 95% ethanol was added to dissolve the dye at the bottom and on the wall and the OD reading at 540 nm was measured to quantify the total biofilm mass. To study the overall effect of the compounds (3.6 mg/mL in 100% ethanol as stock solution), it was added with the inoculation and a time course of biofilm mass was measured. Appropriate amounts of 100% ethanol were added to each sample to eliminate the effect of solvent. Each condition had 3-4 replicates on each plate and was performed over multiple days.

The compounds tested had no inhibitory effect on the growth of either strain of E. coli when compared to controls, demonstrating that these compounds are not antibacterial compounds. Asiatic acid inhibited biofilm formation of the UT189 strain by about 90%, 50%, 15%, and 10% as compared to the controls at 32, 16, 8, and 4 ug/ml, respectively. Corosolic acid inhibited biofilm formation of the UT189 strain by about 85% at 20 ug/ml. Asiatic acid inhibited biofilm formation of the JM109 strain by about 80% and 70% as compared to the controls at 10 and 5 ug/ml, respectively. Madecassic acid inhibited biofilm formation of the JM109 strain by about 75% and 60% as compared to the controls at 10 and 5 ug/ml, respectively. These experiments confirm that asiatic acid, corosolic acid, madecassic acid, and the compounds of the invention inhibit the formation of biofilms against clinical and laboratory strains of E. coli.

### Example 2

Inhibition of Biofilm Formation by Pygenic acid A, Pygenic acid B, 3-acetyl oleanolic acid and Echinocystic acid: Pseudomonas aeruginosa PA01 Assays.

An overnight culture of P. aeruginosa PA01 in LB+1% citrate was prepared. It was incubated at 37° C. shaker for 24 hours. A 1:20 dilution of the overnight culture was prepared. Test compounds are diluted appropriately with a volume of ethanol followed by shaking for approximately 5 minutes and then diluted with an appropriate volume of water.

Replicate 96-well plates are prepared, preferably two to four replicates, with appropriately diluted overnight culture and test compound in each well. Preferably, test compounds are prepared at 10 to 30 micrograms per milliliter. On each 96-well plate controls are appropriately prepared with at least one set of controls consisting of overnight culture and ethanol/water diluent and another set of controls consisting of growth media and ethanol/water diluent. Plates are covered with foil at room temperature and shaken for approximately 24 hours.

After shaking absorbance of the wells of the plates are determined, preferably at 630 nanometers. Liquid is than aspirated out of the wells and each well is filled with diluted crystal violet, preferably approximately 250 microliters of a 1:4 dilution, and allowed to stand for approximately 10 minutes. Each well of the plate is washed, preferably four times, with PBS with approximately one minute between each wash. After the final wash is aspirated out, the plate may be turned over onto paper towels to dump out excess PBS. 95% ethanol is added to each well, preferably 250 microliters. Absorbance of each well of a plate is determined, preferably at 540 nm. Preferably slight shaking is performed during the absorbance reading for approximately 5 minutes. Each well of the plate is than diluted 1 to 50 with ethanol in a separate plate, preferably 145 ul of 95% ethanol and 5 uL from the original plate, and absorbance is determined. Preferably, slight shaking for approximately 3 minutes is performed.

Biofilm inhibition in each well is determined by subtracting the absorbance of the wells with test compounds from wells with controls containing overnight culture subtracting out controls with only media. A typical positive result of biofilm inhibition confirmed with replicates would be 30 percent to 80 percent less biofilm in the wells with test compound compared to the wells with controls of overnight culture.

Pygenic acid A, pygenic acid B, echinocystic acid, 3-acetyl oleanolic acid, and 3-hydroxy-12, 20(30) ursadienoic acid had no inhibitory effect on the growth of P. aeruginosa PA01 when compared to controls. However, pygenic acid A, pygenic acid B, echinocystic acid, 3-acetyl oleanolic acid, and 3-hydroxy-12, 20(30) ursadienoic acid inhibited bioflm formation of P. aeruginosa PAO1 by about 40%, 30%, 25%, 50%, and 40% respectively as compared to the controls at approximately 10 ug/ml. This result clearly demonstrates that pygenic acid A, pygenic acid B, echinocystic acid, 3-acetyl oleanolic acid, and 3-hydroxy-12, 20(30) ursadienoic acid inhibit P. aeruginosa PAO1 biofilm formation but do not inhibit P. aeruginosa PAO1 growth.

### Example 3

A General Procedure for Isolating Compounds of Interest from Plant Samples

For purposes of illustration, the following provides a non-limiting example of a general procedure that may be employed to isolate and purify the compounds described herein. First, an extraction step may be carried out by grinding dried plant material to a homogenous powder and sonicating the powder in an organic solvent, such as a mixture of ethanol: ethanol acetate (50 EtOH: 50 EtOac), and shaking the resulting mixture vigorously for exhaustive extractions. Next, flash chromatographic separation may be carried out by dissolving the organic extract in 5 mL of a solvent, such as methanol: ethyl acetate (MeOH:EtOAc) (50:50), adsorbing it onto silica powder and bringing the dried powder onto a silica column and eluting on the flash chromatography system using a step gradient of (1) 75% hexanes, 25% ethyl acetate, (2) 50% hexanes, 50% ethyl acetate, (3) 100% ethyl acetate, (4) 75% ethyl acetate, 25% methanol, and (5) 50% ethyl acetate, 50% methanol. The flash fraction containing highly lipophilic material may be discarded, like in fraction 1 of 75% hexanes, 25% ethyl acetate, whereas the remaining fractions may be dried, such as by rotary evaporation. Preferably, flash chromatography fraction 3 of 100% ethyl acetate, which shall now be referred to as FCF3 in this specification, will contain the compounds described herein or structurally related compounds of the pentacyclic triterpenes of the ursane or oleane families. More preferably, these compounds will have a carboxylic acid at position 28 and a single, unsubstituted methyl at positions 25, 26, 27, 29, and 30 with a single unsubstituted or substituted methyl at positions 23 and 24.

Preparative HPLC separation may then be carried out. The flash fraction material may be dissolved into either methanol: ethyl acetate (70:30) or 100% methanol (and filtered when necessary). Preferably, FCF3 will be dissolved in methanol: ethyl acetate (70:30). The fractions may be further separated into several individual fractions, such as 40, using a device such as a parallel four-channel preparative HPLC system. A different gradient may be applied to each flash fraction for adequate separation. For example, a first fraction may be eluted in 40-80% acetonitrile in water; a second in 30-70% acetonitrile in water; a third in 20-60% acetonitrile in water; a fourth in 10-50% acetonitrile in water; and so on, with each of these HPLC methods ending with 95% acetonitrile in water for approximately three to seven minutes to elute any remaining compounds. Preferably, FCF3 will be eluted with 30-70% acetonitrile in water using a C18 preparative column (20 mm×100 mm). Preferably, one minute HPLC fractions will be collected into tubes using an automated fraction collector. Of course, those of ordinary skill in the art will appreciate that other organic solvents, and combinations, gradients, and ratios thereof, may be used during HPLC separation—which may depend on the nature of the plant extracts, extraction procedures employed, desired compound, and others.

The resulting HPLC fractions may be dried in an evaporator. The HPLC fractions may then be transferred to plates, such as 96-deep-well plates, using a liquid handling system (e.g., Packard MultiProbe II). These procedures have been generally described by Eldridge, et al. (Anal. Chem. 2002, 74, p. 3963-3971). Preferably, the preparative HPLC fractions collected between 10 minutes to 40 minutes generated from FCF3 will contain the compounds described herein and structurally related compounds of the pentacyclic triterpenes of the ursane or oleane families. More preferably, preparative HPLC fractions between 15 minutes to 38 minutes will contain the compounds described herein or structurally related compounds. Next, the mass or molecular weights of the materials in the samples may be determined using a parallel eight-channel liquid chromatography electrospray detection mass spectrometry (LC-ELSD-MS) system with chromatographic conditions of 5% acetonitrile in water for the first minute, a linear gradient of acetonitrile from 5% to 95% in eight minutes, followed by 95% acetonitrile in water for a minute. Under such chromatographic conditions, the column is equilibrated at 5% acetonitrile in water after each analysis. These procedures have been recently described by Cremin, et al. (Anal Chem.2002). Preferably, electrospray ionization is operated in negative mode. Preferably, the compounds of the invention will have a range of molecular weights from approximately 450 Daltons to approximately 700 Daltons. More preferably, the compounds of the invention will have a range of molecular weights from 456 Daltons similar to ursolic acid to approximately 648 Daltons similar to 2,3-Dihydroxy-3-O-(3-hydroxy, 4-methoxy-cinnamoyl)-ursolic acid. In addition to using molecular ions to locate the compounds of the invention in the preparative HPLC fractions, the HPLC fractions can be screened for biofilm inhibition activity in assays described in the examples of the specification or nuclear magnetic resonance (NMR) spectroscopy spectra can be acquired on the HPLC fractions containing sufficient quantities of material searching for characteristic chemical shifts of the compounds described herein or structurally related compounds of the pentacyclic triterpenes of the ursane or oleane families from 2.5 ppm to 0.7 ppm. These characteristic chemical shifts include signals for each methyl on the pentacyclic structure. More preferably, these HPLC fractions can be further purified by HPLC prior to NMR acquisition. Data processing for determining the appropriate dilution for each sample for normalization may automated with computer software to extract all graphic information, such as retention times, mass spectra, and peak integrations, and to convert such information to text to allow it to be transferred to a database for storage and analysis. The structure of the desired compound may optionally be confirmed using NMR.

### Example 4

Antibacterial effect of Asiatic acid on Haemophilus influenzae (ATCC 10211), E. coli (ATCC 25922), and P. aeruginosa (ATCC 27853)

Using the appropriate NCCLS procedures, the antibacterial effect of asiatic acid on Haemophilus influenzae (ATCC 10211), E. coli (ATCC 25922), and P. aeruginosa (ATCC 27853) was studied at 64 μg/mL. Asiatic acid had no inhibitory effect represented by a MIC (minimal inhibitory concentration) of greater than 64 μg/ml. These results along with the results described in Example 2, further supports that asiatic acid is not an antibacterial compound.

### Example 5

Effect of Asiatic Acid on Mature Biofilms of clinical isolates of P. aeruginosa

Clinical isolates of P. aeruginosa from cystic fibrosis patients were passed twice on tryptic soy agar with 5% sheep blood after retrieval from −80° C. and then grown overnight in CAMHB. After dilution of a culture to 0.5 McFarland in broth medium, 100 μl was transferred in triplicate to wells of a flat-bottom 96-well microtiter plate. Bacterial biofilms were formed by immersing the pegs of a modified polystyrene microtiter lid into this biofilm growth plate, followed by incubation at 37° C. for 20 hours with no movement.

Peg lids were rinsed three times in sterile water, placed onto flat-bottom microtiter plates containing biofilm inhibitors at 5 ug/ml in 100 μl of CAMHB per well and incubated for approximately 40 hours at 37° C.

Pegs were rinsed, placed in a 0.1% (wt/vol) crystal violet solution for 15 min, rinsed again, and dried for several hours. To solubilize adsorbed crystal violet, pegs were incubated in 95% ethanol (150 μl per well of a flat-bottom microtiter plate) for 15 min. The absorbance was read at 590 nm on a plate reader. The wells containing asiatic acid were compared to negative controls. Negative controls were prepared as stated above but without asiatic acid.

Asiatic acid caused an average detachment of mature biofilms of approximately 50% at 5 ug/ml compared to the negative controls against eighteen clinical isolates of P. aeruginosa. The range of detachment of mature biofilms against all eighteen clinical isolates was 25% to 74%. This example demonstrates the ability of asiatic acid and the compounds of the invention to reduce mature biofilms in clinical isolates of P. aeruginosa.

### Example 6

Effect of Asiatic acid, Corosolic acid, or Madecassic acid in combination with Tobramycin on Biofilm formation of Pseudomonas aeruginosa.

Biofilm formation of P. aeruginosa was evaluated using a standardized biofilm method with a rotating disk reactor (RDR). This method provides a model resembling the formation of biofilms in cystic fibrosis patients. The rotating disk reactor consists of a one-liter glass beaker fitted with a drain spout. The bottom of the vessel contains a magnetically driven rotor with six 1.27 cm diameter coupons constructed from polystyrene. The rotor consists of a star-head magnetic stir bar upon which a disk was affixed to hold the coupons. The vessel with the stir bar was placed on a stir plate and rotated to provide fluid shear. A nutrient solution (AB Trace Medium with 0.3 mM glucose, see Table 3 below for composition) was added through a stopper in the top of the reactor at a flow rate of 5 ml/min. The reactor volume was approximately 180 ml and varied slightly between reactors depending on the placement of the drain spout and the rotational speed of the rotor. At a volume of 180 ml, the residence time of the reactors was 36 minutes. The reactors were operated at room temperature (c.a. 26° C.).

TABLE 3
Composition of the AB Trace Medium used for the RDR test.

| | | Concentration |
|---|---|---|
| Component | Formula | (g/l) |
| Disodium phosphate | Na₂HPO₄ | 6.0 |
| Monopotassium | KH₂PO₄ | 3.0 |
| phosphate | | |
| Sodium Chloride | NaCl | 3.0 |
| Ammonium sulfate | (NH₄)₂SO₄ | 2.0 |
| Magnesium chloride | MgCl₂ | 0.2 |
| Glucose | C₆O₁₂H₆ | 0.054 |
| Calcium chloride | CaCl₂ | 0.010 |
| Sodium sulfate | Na₂SO₄ | 0.011 |
| Ferric chloride | FeCl₃ | 0.00050 |

For each test, two RDRs were operated in parallel with one receiving test compound and the other serving as an untreated control. The RDRs were sterilized by autoclave, then filled with sterile medium and inoculated with P. aeruginosa strain PAO1. The reactors were then incubated at room temperature in batch mode (no medium flow) for a period of 24 hours, after which the flow was initiated for a further 24 hour incubation. Test compounds were dissolved in 10 ml ethanol to achieve a concentration of 1.8 mg/ml. After the 48 hours of biofilm development described above, the 10 ml of ethanol containing the test compounds were added to the reactor to achieve a final concentration of approximately 50, 100, or 200 μg/ml. Control reactors received 10 ml of ethanol. The reactors were then incubated for an additional 24 hours in batch (no flow) mode. After this incubation period, the six coupons were removed from each reactor and placed in 12-well polystyrene tissue culture plates with wells containing either 2 ml of a 100 μg/ml tobramycin solution or 2 ml of phosphate-buffered saline (PBS). These plates were incubated at room temperature for two hours. The coupons were then rinsed by three transfers to plates containing 2 ml of fresh PBS. For each two RDR reactors run in parallel, four sets of three coupons were obtained: one set with no test compound treatment and no tobramycin treatment, one set with no test compound treatment and tobramycin treatment, one set treated with a test compound treatment and no tobramycin treatment, and one set treated with a test compound treatment and tobramycin. After rinsing, one coupon of each set of three was stained with a LIVE/DEAD® BacLight™ Bacterial Viability Kit (Molecular Probes, Eugene Oreg.) and imaged using epifluorescent microscopy. The remaining two coupons were placed in 10 ml of PBS and sonicated for five minutes to remove and disperse biofilm cells. The resulting bacterial suspensions were then serially diluted in PBS and plated on tryptic soy agar plates for enumeration of culturable bacteria. The plates were incubated for 24 hours at 37° C. before colony forming units (CFU) were determined.

The treatments of the individual test compounds with and without tobramycin are listed in Table 4. The results are averages from experiments performed on three separate days for each test compound. The values reported are as loglo CFU.

TABLE 4

| | Asiatic | Asiatic | Asiatic | Madecassic | Corosolic |
|---|---|---|---|---|---|
| | acid | acid | acid | acid | acid |
| Test | 50 μg/ml | 100 | 200 | 100 μg/ml | 100 μg/ml |
| Compound | | μg/ml | μg/ml | | |
| Concentration | | | | | |
| Tobramycin | 5.3 | 5.5 | 5.2 | 5.5 | 5.3 |
| and Test | | | | | |
| Compound | | | | | |
| Test | 7.7 | 7.7 | 7.5 | 7.5 | 7.8 |
| Compound | | | | | |
| Tobramycin | 5.8 | 6.5 | 6.1 | 6.7 | 6.2 |
| Control | 7.5 | 7.8 | 7.6 | 8.0 | 8.0 |

The results clearly demonstrate the abilities of asiatic acid, corosolic acid, and madecassic acid to increase the biofilm's susceptibility to tobramycin by modifying the biofilm. In combination with tobramycin these test compounds demonstrated an additional reduction of 67% to 93% CFU when compared to tobramycin alone. This translates into a reduction of approximately 1,000,000 to 4,500,000 cells of P. aeruginosa at 100 μg/ml.

As a comparison to multiple published clinical studies, these results with asiatic acid, corosolic acid, or madecassic acid in combination with tobramycin demonstrate that improved lung function (FEV or forced expiratory volume) and decreased average CFU (density) in sputum from patients with cystic fibrosis would be observed in a combination therapy involving these compounds (Ramsey, Bonnie W. et. al., “Intermittent administration of inhaled tobramycin in patients with cystic fibrosis”, New England J. Medicine 340(1):23-30, 1999; Saiman, L. “The use of macrolide antibiotics in patients with cystic fibrosis”, Curr Opin Pulm Med, 2004, 10:515:523; Pirzada, O. et al. “Improved lung function and body mass index associated with long-term use of Macrolide antibiotics.”, J. Cystic Fibrosis, 2003, 2, p. 69-71). Using the endpoints listed in these publications and used in Cystic Fibrosis clinical trials, this example demonstrates that a combined treatment of tobramycin and a compound of the invention would provide benefit to Cystic Fibrosis patients or other people suffering from chronic lung infections. The research results of this example also demonstrate that the compounds of the invention in combination with an antibiotic would remove biofilms from teeth, skin, tissues, catheters, medical devices, and other surfaces.

### Example 7

Effect of Asiatic acid on Biofilm Growth and Inhibition with Streptococcus mutans 25175 and Streptococcus sobrinus 6715.

Asiatic acid was tested against S. mutans 25175 and S. sobrinus 6715 at a concentration of 40 μg/ml using the method described in Example 1. The use of 1 mL polycarbonate tubes were used in place of 96 well polysterene microtiter plates.

Testing asiatic acid at 40 μg/mL against S. mutans 25175 and S. sobrinus 6715 showed greater than 75% biofilm growth inhibition.

### Example 8

The Effects of Asiatic acid, Corosolic asid, and Ursolic acid on the Binding to and Invasion of E. coli clinical strain UT189 against bladder epithelial cells

The effect of test compounds on bacterial invasion of E. coli clinical strain UT189 was studied as described in Elsinghorst, et al. 1994, Methods Enzymol, 236:405-420; and Martinez et al., 2000, EMBO J., 19:2803-2812. Epithelial bladder cells were grown in plates. Asiatic acid, corosolic acid, or ursolic acid were added at concentrations of 10 μg/ml, 20 μg/ml, or 40 μg/ml to bacteria and epithelial cells for approximately 5, 15, 30, or 60 minutes with approximately 10⁷ CFU of E. coli. Binding was assessed at time zero and invasion was assessed at approximately 5, 15, 30, or 60 minutes from completing the mixture of compound, bacteria, and epithelial cells. As a control ethanol was added to cells to a final concentration of 0.1%. The effect of bacterial viability and bacterial adherence during the infection period was evaluated according to the methods described in Martinez et al., 2000, EMBO J., 19:2803-2812. The test compounds did not affect the binding of E. coli to bladder epithelial cells. The test compounds reduced the invasion of E. coli into bladder epithelial cells.

40 g/ml of corosolic acid with bacteria and epithelial cells for 60, 15, and 5 minutes reduced invasion of E. coli into bladder epithelial cells by 90%, 70%, and 10%, respectively, as compared to the controls. These experiments were performed in triplicate. Furthermore and separately, 40 μg/ml and 20 μg/ml of corosolic acid with bacteria and epithelial cells for 60 minutes reduced invasion of E. coli into bladder epithelial cells by 90% (n=7) and 65% (n=4), respectively, as compared to the controls. These experiments demonstrate a dose and time dependent effect of corosolic acid interrupting the pathogenesis cycle of E. coli. 40 μg/ml of asiatic acid and ursolic acid with bacteria and epithelial cells for 60 minutes reduced invasion of E. coli into bladder epithelial cells by 87% (n=7) and 76% (n=4), respectively.

These experiments demonstrate that corosolic acid, asiatic acid, ursolic acid, and the compounds of the invention reduce invasion and therefore interrupt the pathogenesis of E. coli into bladder epithelial cells. The significance of this effect is magnified when taking into account that the cycle of pathogenesis of E. coli in recurrent urinary tract infections has repeated invasion episodes that are critical for its survival. Also, it has been demonstrated that invasion of E. coli into the bladder epithelial cells enables them to resist the mammalian immune response and subsequently re-invade deeper into tissues. The compounds of the invention therefore interrupt a key point in its life cycle further clarifying the significance of these compounds in treating chronic infections involving biofilms.

### Example 9

Isolation of asiatic acid and madecassic acid were performed as follows. Approximately 150 grams of Centella asiatica was extracted twice overnight with 500 ml EtOAc:EtOH (1:1) and then filtered. The two extracts were combined and the solvent removed via evaporation. Approximately one gram of this extract was separated on a 50 gram silica flash column (9.5 cm×3.7 cm; 55 um; 70A) with a gradient step using hexane:ethyl acetate (75:25), hexane:ethyl acetate (50:50), ethyl acetate (100%). The volume of each flash fraction was 300 ml. The 100% ethyl acetate flash fraction that is referred to herein as FCF3 did contain asiatic acid and madecassic acid. FCF3 was subjected to semi-preparative HPLC on C18 BetaMax Neutral column (250×8 mm; 5 um) using a gradient with water with 0.05% trifluoroacetate (TFA) and acetonitrile with 0.05% TFA, mobile phase A and B respectively. The gradient consisted of 40% B isocratic for 5 min, then from 40% to 60% B in 30 min. Asiatic acid and madecassic acid were then isolated as pure compounds using an automated fraction collector.

### Example 10

Bladder Concentrations of Asiatic Acid and Madecassic Acid in Rats

Pharmacokinetic studies of asiatic acid and madecassic acid in rats were performed separately. Asiatic acid and madecassic acid were evaluated at 50 mg/kg (oral). Two animals were assigned to the each group. Prior to dosing, a baseline blood sample was taken from each animal. At time zero for asiatic acid and madecassic acid, a single bolus dose in 50% Labrasol (Gattefosse) was given to each animal. Bladders were analyzed at 24 hours. Concentrations of both asiatic acid and madecassic acid in the bladder were approximately 30 μg/g at 24 hours. The efficacy of asiatic acid and madecassic acid in the tissues of mammals is further supported by the data in example 7 shows that the compounds of the invention significantly reduce bacterial invasion within 15 minutes.

These experiments demonstrate that asiatic acid and madecassic acid are in adequate concentrations in the bladders of mice to reduce invasion of bacteria and the formation of biofilms.

### Example 11

The Effects of Asiatic acid, Corosolic asid, and Ursolic acid on the Pathogenesis of E. coli clinical strain UT189 in Mice

The procedures in this example has been previously reported by Justice, S. et al. Differentiation and development pathways of uropathogenic Escherichia coli in urinary tract pathogenesis. PNAS, 2004, 101(5), p. 1333-1338. Briefly, E. coli UT189[pCOMGFP] was prepared after retrieval from frozen stocks by inoculating appropriately in LB medium statically for approximately 20 hours. Cells were harvested and suspended in 1 ml of PBS. Cells were diluted appropriately to achieve approximately a 10⁸ CFU or 10⁷ CFU input into C3H/HeN mice (2 mice per group).

Mice were deprived of water for approximately two hours. In experiment 1, all mice were anesthetized with 0.15 cc ketamine cocktail. In experiment 2, all mice were anesthetized with isofluorane. In experiment 1, urine was dispelled from the bladders and approximately 40 μg/ml of test compound or an appropriate amount of ethanol as control was introduced into the bladders via catheterization of the urethra using a tubing coated tuberculin syringe. 30 minutes was allowed to elapse. In experiment 2, bladders were not pre-incubated with test compounds. Bladders were then expelled and an inoculum of 10⁸ CFU (Experiment 1) or 10⁷ CFU (Experiment 2) of E. coli containing 40 μg/ml of test compound or equivalent amount of ethanol as controls were introduced into the bladders as indicated above.

In experiment 1 five hours elapsed and in experiment 2 six hours elapsed, and then mice were anesthetized and sacrificed appropriately. The bladders were removed, bisected, stretched, and fixed in 3% paraformaldehyde for 1 hour at room temperature. Bladders were then permeabilized in 0.01% Triton/PBS for 10 minutes and counter stained with TOPRO3 (Molecular Probes) for 10 minutes for visualization by confocal microscopy. Bladders were mounted on Prolong antifade (Molecular Probes).

In experiment 1, corosolic acid, asiatic acid, and ursolic acid demonstrated a 94%, 77%, and 70% reduction, respectively, in biofilm pods or IBCs in the bladders of mice as compared to the controls by examination with confocal microscopy. In experiment 2, both corosolic acid and asiatic acid demonstrated approximately a 60% reduction in large biofilm pods or large IBCs in the bladders of mice as compared to the controls by examination with confocal microscopy.

The results of these experiments demonstrate that the compounds of the invention interrupt the pathogenesis of clinical strains of E. coli in mice. Moreover, it becomes readily apparent the significant impact the compounds of the invention will have on treating chronic infections involving biofilms from the understanding described by Justice, S, et al. that biofilm pods or IBCs play an integral role in the recurrence of urinary tract infections (Justice, S. et al. Differentiation and development pathways of uropathogenic Escherichia coli in urinary tract pathogenesis. PNAS, 2004, 101(5), p. 1333-1338). In this publication the authors describe and their experiments demonstrate that IBCs prevent the mammalian immune response from eradicating the bacterial population and enable them to increase their numbers. Therefore, disabling this advantage, or interrupting the pathogenesis of bacteria, the compounds of the invention work in combination with a mammalian immune response or an antibiotic, as demonstrated in other examples in this specification, to reduce, prevent, treat, or eradicate infections involving biofilms. Furthermore, this animal model is representative of chronic lung, ear, and sinus infections, acne, rosacea, and chronic wounds. It is also representative of the cycle of pathogenesis of other E. coli infections such as, but not limited to, pyelonephritis, prostatitis, meningitis, sepsis, and gastrointestinal infections.

### Example 12

The Effects of Asiatic Acid on the Pathogenesis of E. coli Clinical Strain UT189 in Mice

The procedures in this example has been previously reported by Justice, S. et al. Differentiation and development pathways of uropathogenic Escherichia coli in urinary tract pathogenesis. PNAS, 2004, 101(5), p. 1333-1338. Briefly, E. coli UTI89[pCOMGFP] was prepared after retrieval from frozen stocks by inoculating appropriately in LB medium statically for approximately 20 hours. Cells were harvested and suspended in 1 ml of PBS. Cells were diluted appropriately to achieve approximately 10⁷ CFU input into C3H/HeN mice.

Mice were deprived of water for approximately two hours. All mice were anesthetized with isofluorane. Urine was dispelled from the bladders and an inoculum of approximately 10⁷ CFU of E. coli were introduced into the bladders as indicated above. Treatments of sulfamethoxazole and trimethoprim (SMZ/TMP), asiatic acid, and combination of SMZ/TMP and asiatic acid was evaluated.

2 mice did not receive asiatic acid or SMZ/TMP during the experiment. 3 mice received asiatic acid by orally at approximately 25 milligram per kilogram twice a day beginning one day prior to infection each day during the experiment. Asiatic acid was prepared in 50% Labrasol®. 5 mice received SMZ/TMP in their drinking water at a concentration of 270 micrograms of SMZ per millilter and 54 micrograms of TMP per milliliter immediately after infection throughout the experiment. 5 mice received asiatic acid and SMZ/TMP in combination dosed according to the individual dosing groups. The experiment was performed for approximately 2 days after inoculation. Mice were anesthetized and sacrificed appropriately. The bladders were removed and colony forming units (CFU) were determined as previously described by Justice, S. et al.

The bladder CFU for the control, SMZ/TMP, asiatic acid, and combination groups were 4.7×10⁶, 6.7×10⁴, 2.9×10³, and 2.3×10⁴, respectively. Asiatic acid was superior to SMZ/TMP at preventing the colonization of bladders. The results of this experiment demonstrate that the compounds of the invention can be delivered orally to interrupt the pathogenesis of clinical strains of E. coli in mice. This experiment also further demonstrates the compounds of the invention may be superior to conventional antibiotics.

### Example 13

A topical gel was prepared containing 2% of madecassic acid by weight with azithromycin for use in treating acne, rosacea, and skin infections.

0.25 gram of madecassic acid was dissolved in 6.75 grams of ethanol. 0.2 grams of azithromycin was dissolved in this solution. 0.25 grams of hydroxypropyl methylcellulose was added with gentle stirring until a homogenous solution was obtained. 4.8 grams of water was then added with gentle shaking.

This formulation was stored for thirty days at 2° C. to 8° C., room temperature (approximately 22° C.), and at 30° C. It remained homogenous for thirty days at each storage condition. A formulation without antibiotic could also be prepared using this same procedure.

### Example 14

Madecassic Acid, Pharmaceutical Formulation for Nebulization

Solutions were prepared comprising 2 mg/ml and 10 mg/ml of madecassic acid in ethanol/propylene glycol/water (85:10:5). These solutions were nebulized separately by a ProNeb Ultra nebulizer manufactured by PARI. The nebulized solutions were collected in a cold trap, processed appropriately, and detected by mass spectrometry. Madecassic acid was recovered from both formulations demonstrating that nebulization can be used to deliver this compound to patients with lung infections.

### Example 15

Madecassic Acid, 2% Toothpaste Formulation

Toothpaste preparations were prepared containing 2% madecassic acid with and without antibiotic and with and without polymer. Polymer, Gantrez® S-97, was added to improve retention of madecassic acid and antibiotic on teeth.

All of the dry ingredients were mixed together. Glycerin was slowly added while mixing. An aliquot of water was added slowly and thoroughly mixed. Peppermint extract was added and then the rest of the water was added while mixing. Madecassic acid and antibiotic were then added until homogenous.

Formulation A

| Ingredients | Parts By Weight |
|---|---|
| Sorbitol | 20.0 |
| Glycerin | 22.0 |
| Silica | 20 |
| Sodium lauryl sulfate | 2.0 |
| Xanthum gum | 1 |
| Madecassic Acid | 2.0 |
| Peppermint extract | 1.0 |
| Sodium fluoride | 0.3 |
| Water | 31.7 |

Formulation B

| Ingredients | Parts By Weight |
|---|---|
| Sorbitol | 20.0 |
| Glycerin | 22.0 |
| Silica | 20 |
| Sodium lauryl sulfate | 2.0 |
| Xanthum gum | 1 |
| Madecassic Acid | 2.0 |
| Triclosan | 0.3 |
| Peppermint extract | 1.0 |
| Sodium fluoride | 0.3 |
| Gantrez ® S-97 | 2.5 |
| Water | 28.9 |

Formulations A and B were prepared and stored for thirty days at 2° C. to 8° C., room temperature (approximately 22° C.), and at 30° C.

What is claimed:

1. A pharmaceutical composition comprising an antimicrobial agent, more than 1% by weight a compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof, and a pharmaceutically acceptable carrier.

2. The composition of claim 1, wherein the pharmaceutically acceptable carrier is one that permits administration of the pharmaceutical composition orally, topically, rectally, percutaneously, by parenteral injection, intranasally or by inhalation.

3. The composition of claim 1, wherein the composition is a topical preparation.

4. The composition of claim 3, wherein the topical preparation is a gel, cream, emollient, or soap.

5. The composition of claim 1, wherein the composition is a dentifrice.

6. The composition of claim 5, wherein the composition is a dentifrice selected from the group consisting of toothpaste, toothpowder, liquid dentifrice, mouth detergent, mouthwash, troches, chewing gum, dental or gingival massage cream, dental strip, dental gel, and gargle tablet.

7. The composition of claim 5, wherein the dentifrice is a tooth paste and further comprises a tooth or gum adherence promoting substance selected from the group consisting of copolymers of methyl vinyl ether and maleic anhydride, copolymers of vinyl pyrrolidone and vinyl acetate, and cyclodextrins.

8. The composition of claim 5, wherein the antimicrobial agent is selected from the group consisting of triclosan, metronidazole, tetracyclines, quinolones, plant essential oils, camphor, thymol, carvacrol, menthol, eucalyptol, and methyl salicylate.

9. The composition of claim 1, wherein the composition is an oral dose form selected from the group consisting of a capsule, tablet, or liquid, and wherein the compound makes up more than 1% by weight of the composition.

10. The composition of claim 7, wherein the antimicrobial agent is an antibiotic.

11. The composition of claim 8, wherein the antibiotic is selected from the group consisting of tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, and aminoglycosides.

12. The composition of claim 1, wherein the compound makes up from about 2% to about 60% by weight of the composition.

13. The composition of claim 12, wherein the compound makes up about 2% to about 5% by weight of the composition.

14. The composition of claim 13, wherein the compound makes up about 2% by weight of the composition.

15. The composition of claim 1, wherein the compound is selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof.

16. The composition of claim 15, wherein the compound is selected from the group consisting of asiatic acid, madecassic acid, corosolic acid and salts, hydrates, solvates, prodrugs and N-oxides thereof.

17. The composition of claim 17, wherein the compound is corosolic acid or a salt, hydrate, solvate, prodrug or N-oxide thereof.

18. A pharmaceutical composition comprising an antimicrobial agent, one and only one compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof, and a pharmaceutically acceptable carrier, and wherein the compound is present in a concentration of at least about 0.1% by weight, based on the total weight of the composition.

19. The composition of claim 18, wherein the pharmaceutically acceptable carrier is one that permits administration of the pharmaceutical composition orally, topically, rectally, percutaneously, by parenteral injection, intranasally or by inhalation.

20. The composition of claim 19, wherein the composition is a topical preparation.

21. The composition of claim 20, wherein the topical preparation is a gel, cream, emollient, or soap.

22. The composition of claim 18, wherein the composition is a dentifrice.

23. The composition of claim 22, wherein the composition is a dentifrice selected from the group consisting of toothpaste, toothpowder, liquid dentifrice, mouth detergent, mouthwash, troches, chewing gum, dental or gingival massage cream, dental strip, dental gel, and gargle tablet.

24. The composition of claim 22, wherein the dentifrice is a tooth paste and further comprises a tooth or gum adherence promoting substance selected from the group consisting of copolymers of methyl vinyl ether and maleic anhydride, copolymers of vinyl pyrrolidone and vinyl acetate, and cyclodextrins.

25. The composition of claim 22, wherein the antimicrobial agent is selected from the group consisting of triclosan, metronidazole, tetracyclines, quinolones, plant essential oils, camphor, thymol, carvacrol, menthol, eucalyptol, and methyl salicylate.

26. The composition of claim 18, wherein the composition is an oral dose form selected from the group consisting of a capsule, tablet, or liquid, and wherein the concentration of the compound is greater than about 0.1% by weight.

27. The composition of claim 18, wherein the antimicrobial agent is an antibiotic.

28. The composition of claim 27, wherein the antibiotic is selected from the group consisting of tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, and aminoglycosides.

29. The composition of claim 18 wherein the concentration of the compound is from about 2% to about 60% by weight.

30. The composition of claim 29, wherein the concentration of the compound is from about 2% to about 5% by weight.

31. The composition of claim 30, wherein the concentration of the compound is from about 2% by weight.

32. The composition of claim 18, wherein the compound is selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof.

33. The composition of claim 32, wherein the essentially pure compound is selected from the group consisting of asiatic acid, madecassic acid, corosolic acid and salts, hydrates, solvates, prodrugs and N-oxides thereof.

34. The composition of claim 33, wherein the compound is corosolic acid or a salt, hydrate, solvate, prodrug or N-oxide thereof.

35. A pharmaceutical composition comprising an antimicrobial agent, at least 0.1% by weight a compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20 P-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof and a pharmaceutically acceptable carrier, wherein said pharmaceutical composition contains no or less than 1.0% by weight triterpene glycoside, no or less than 1.0% by weight fatty acid, no or less than 1.0% by weight sterol, or no or less than 1.0% by weight hydrocarbon.

36. The composition of claim 35, wherein the pharmaceutically acceptable carrier is one that permits administration of the pharmaceutical composition orally, topically, rectally, percutaneously, by parenteral injection, intranasally or by inhalation.

37. The composition of claim 35, wherein the composition is a topical preparation.

38. The composition of claim 37, wherein the topical preparation is a gel, cream, emollient, or soap.

39. The composition of claim 35, wherein the composition is a dentifrice.

40. The composition of claim 39, wherein the composition is a dentifrice selected from the group consisting of toothpaste, toothpowder, liquid dentifrice, mouth detergent, mouthwash, troches, chewing gum, dental or gingival massage cream, dental strip, dental gel, and gargle tablet.

41. The composition of claim 40, wherein the dentifrice is a tooth paste and further comprises a tooth or gum adherence promoting substance selected from the group consisting of copolymers of methyl vinyl ether and maleic anhydride, copolymers of vinyl pyrrolidone and vinyl acetate, and cyclodextrins.

42. The composition of claim 39, wherein the antimicrobial agent is selected from the group consisting of triclosan, metronidazole, tetracyclines, quinolones, plant essential oils, camphor, thymol, carvacrol, menthol, eucalyptol, and methyl salicylate.

43. The composition of claim 35, wherein the composition is an oral dose form selected from the group consisting of a capsule, tablet, or liquid, and wherein the compound makes up more than 1% by weight of the composition.

44. The composition of claim 43, wherein the compound is selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof.

45. The composition of claim 44, wherein the compound is corosolic acid or a salt, hydrate, solvate, prodrug or N-oxide thereof.

46. The composition of claim 35, wherein the antimicrobial agent is an antibiotic.

47. The composition of claim 46, wherein the antibiotic is selected from the group consisting of tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, and aminoglycosides.

48. The composition of claim 35, wherein the compound makes up about 2% to about 60% by weight of the composition.

49. The composition of claim 48, wherein the compound makes up about 2% to about 5% by weight of the composition.

50. The composition of claim 49, wherein the compound makes up about 2% by weight of the composition.

51. An method for preventing, inhibiting or reducing a biofilm or its formation comprising contacting the biofilm or a cell capable of biofilm formation with an effective amount of a composition comprising a compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof, and an acceptable carrier, thereby preventing, inhibiting or reducing the biofilm or its formation.

52. The method of claim 51, wherein the biofilm or biofilm formation is prevented, inhibited or reduced in vivo and the acceptable carrier is a pharmaceutically acceptable carrier.

53. The method of claim 51, wherein the biofilm or a cell capable of biofilm formation is contacted by applying a dentifrice is selected from the group consisting of toothpaste, toothpowder, liquid dentifrice, mouth detergent, mouthwash, troches, chewing gum, dental or gingival massage cream, dental strip, dental gel, and gargle tablet.

54. The method of claim 53, wherein the dentifrice is a tooth paste and further comprises a tooth or gum adherence promoting substance selected from the group consisting of copolymers of methyl vinyl ether and maleic anhydride, polyvinylmethyl ether maleic acid, copolymers of vinyl pyrrolidone and vinyl acetate, and cyclodextrins.

55. The method of claim 53, wherein the antimicrobial agent is selected from the group consisting of triclosan, metronidazole, tetracyclines, quinolones, plant essential oils, camphor, thymol, carvacrol, menthol, eucalyptol, and methyl salicylate.

56. The method of claim 51, wherein the biofilm or cell capable of biofilm formation is associated with acne and wherein the biofilm or a cell capable of biofilm formation is contacted by applying a topical preparation that is a gel, cream, emollient, or soap.

57. The method of claim 51, wherein the composition further comprises an antibiotic.

58. The method of claim 57, wherein the antibiotic is selected from the group consisting of tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, and aminoglycosides.

59. The method of claim 51, wherein the biofilm or biofilm formation is inhibited or prevented on a substrate.

60. The method of claim 59, wherein the substrate is a biological structure selected from the group consisting of a regenerating protein of a mammalian cellular membranes, dental enamel, gum, tongue, and biological polymer.

61. The method of claim 59, wherein the substrate is a medical device selected from the group consisting of a central venous catheter, urinary catheter, endotracheal tube, mechanical heart valve, pacemaker, vascular graft, stent, and prosthetic joint.

62. The method of claim 51, wherein said compound contains a C-3 hydroxyl group and wherein said prodrug of said compound is selected from the group consisting of esters, sulfonates, and carbonates of said C-3 hydroxyl group.

63. The method of claim 51, wherein said compound contains a C-28 carboxyl group and wherein said prodrug of said compound is selected from the group consisting of esters, amides, and hydrazides of said C-28 carboxyl group.

64. The method of claim 51, wherein the biofilm or cell capable of biofilm formation is associated with a respiratory infection and wherein the biofilm or a cell capable of biofilm formation is contacted by administering the composition by an inhaler or nebulizer.

65. The method of claim 64, wherein the respiratory infection occurs in a patient with cystic fibrosis.

66. The method of claim 51, wherein the compound is selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof.

67. The method of claim 66, wherein the compound is selected from the group consisting of asiatic acid, madecassic acid, corosolic acid and salts, hydrates, solvates, prodrugs and N-oxides thereof.

68. The method of claim 67, wherein the compound is corosolic acid or a salt, hydrate, solvate, prodrug or N-oxide thereof.

69. A method for preventing, inhibiting or reducing a biofilm or its formation comprising contacting the biofilm or a cell capable of biofilm formation with an effective amount of a compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof, thereby preventing, inhibiting or reducing the biofilm or its formation.

70. The method of claim 69, wherein the biofilm or biofilm formation is inhibited or prevented on a substrate.

71. The method of claim 70, wherein the substrate is a biological structure selected from the group consisting of a regenerating protein of a mammalian cellular membranes, dental enamel, gum, tongue, and biological polymer.

72. The method of claim 71, wherein the prevention, inhibition or reduction of biofilm formation is effected in a vessel hull, car, airplane, industrial equipment, device, membrane, filter, microtiter plate, continuous flow chamber, or piece of machinery.

73. The method of claim 69, wherein said compound contains a C-3 hydroxyl group and wherein said prodrug of said compound is selected from the group consisting of esters, sulfonates, and carbonates of said C-3 hydroxyl group.

74. The method of claim 69, wherein said compound contains a C-28 carboxyl group and wherein said prodrug of said compound is selected from the group consisting of esters, amides, and hydrazides of said C-28 carboxyl group.

75. The method of claim 69, wherein the compound is selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof.

76. The method of claim 75, wherein the compound is selected from the group consisting of asiatic acid, madecassic acid, corosolic acid and salts, hydrates, solvates, prodrugs and N-oxides thereof.

77. The method of claim 76, wherein the compound is corosolic acid or a salt, hydrate, solvate, prodrug or N-oxide thereof.

78. A method for treating, controlling, reducing or preventing a bacterial infection in a subject in need thereof by administering to the subject an effective amount of a composition comprising a compound selected from the group consisting of 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof and an acceptable carrier, thereby treating, controlling, reducing or preventing the bacterial infection.

79. The method of claim 78, wherein the composition is administered to the subject orally, topically, rectally, percutaneously, by parenteral injection, intranasally or by inhalation.

80. The method of claim 79, wherein the infection is a chronic bacterial infection.

81. The method of claim 80, wherein the chronic bacterial infection is selected from the group consisting of urinary tract infection, gastritis, lung infection, ear infection, cystitis, pyelonephritis, arterial damage, leprosy, tuberculosis, benign prostatic hyperplasia, prostatitis, osteomyelitis, bloodstream infection, cirrhosis, skin infection, acne, rosacea, open wound infection, chronic wound infection, and sinus infection.

82. The method of claim 80, wherein the chronic bacterial infection causes an autoimmune disease.

83. The method of claim 78, wherein the subject is a mammal and the carrier is a pharmaceutically acceptable carrier.

84. The method of claim 83, wherein the mammal is a human.

85. The method of claim 78, wherein the compound makes up about 0.1% to about 60.0% by weight of the composition.

86. The method of claim 85 wherein the compound makes up about 0.1% to about 5.0% by weight of the composition.

87. The method of claim 86, wherein the compound makes up about 2% by weight of the composition.

88. The method of claim 78, wherein the composition is a topical preparation.

89. The method of claim 88, wherein the topical preparation is a gel, cream, emollient, or soap.

90. The method of claim 78, wherein the composition is a dentifrice.

91. The method of claim 90, wherein the composition is a dentifrice selected from the group consisting of toothpaste, toothpowder, liquid dentrifrice, mouth detergent, mouthwash, troches, chewing gum, dental or gingival massage cream, dental strip, dental gel, and gargle tablet.

92. The method of claim 91, wherein the dentifrice is a tooth paste and further comprises a tooth or gum adherence promoting substance selected from the group consisting of copolymers of methyl vinyl ether and maleic anhydride, polyvinylmethyl ether maleic acid, copolymers of vinyl pyrrolidone and vinyl acetate, and cyclodextrins.

93. The method of claim 92, wherein the antimicrobial agent is selected from the group consisting of triclosan, metronidazole, tetracyclines, quinolones, plant essential oils, camphor, thymol, carvacrol, menthol, eucalyptol, and methyl salicylate.

94. The method of claim 93, wherein the composition is an oral dose form selected from the group consisting of a capsule, tablet, or liquid, and wherein the compound makes up more than 1% by weight of the composition.

95. The method of claim 94, wherein the composition further comprises an antimicrobial agent.

96. The method of claim 95, wherein the antimicrobial agent is an antibiotic.

97. The method of claim 96, wherein the antibiotic is selected from the group consisting of tobramycin, clindamycin, ciprofloxacin, tetracyclines, rifampin, triclosan, oxfloxacin, macrolides, penicillins, cephalosporins, amoxicillin/clavulanate, quinupristin/dalfopristin, amoxicillin/sulbactum, metronidazole, fluoroquinolones, quinolones, ketolides, and aminoglycosides.

98. The method of claim 78, wherein said compound contains a C-3 hydroxyl group and wherein said prodrug of said compound is selected from the group consisting of esters, sulfonates, and carbonates of said C-3 hydroxyl group.

99. The method of claim 78, wherein said compound contains a C-28 carboxyl group and wherein said prodrug of said compound is selected from the group consisting of esters, amides, and hydrazides of said C-28 carboxyl group.

100. The method of claim 78, wherein the compound is selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof.

101. The method of claim 100, wherein the compound is selected from the group consisting of asiatic acid, madecassic acid, corosolic acid and salts, hydrates, solvates, prodrugs and N-oxides thereof.

102. The method of claim 101, wherein the compound is corosolic acid or a salt, hydrate, solvate, prodrug or N-oxide thereof.

103. A process for making a pharmaceutical composition, comprising combining an essentially pure preparation of an antimicrobial agent, an essentially pure preparation of a compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin and salts, hydrates, solvates, prodrugs and N-oxides thereof, and a pharmaceutically acceptable carrier to form the pharmaceutical composition.

104. The process of claim 103, wherein the essentially pure antimicrobial agent is obtained from any one of a commercial source, a natural source, or by direct synthesis.

105. The process of claim 104, wherein the natural source is a plant, a plant cell or culture media derived thereof.

106. The process of claim 105, wherein the natural source is a microorganism or culture media derived thereof.

107. The process of claim 106, wherein the essentially pure compound is obtained from any one of a commercial source, a natural source, or by direct synthesis.

108. The process of claim 107, wherein the natural source is a plant, a plant cell or culture media derived thereof.

109. The process of claim 108, wherein the essentially pure compound is obtained by a process comprising the steps of:
a) Extracting the plant or plant cell derived material in an organic solvent;
b) Adsorbing the extract from (a) on a powder;
c) Subjecting the powder from (c) to flash chromatographic separation step gradients of (i) 75% hexanes, 25% ethyl acetate, (ii) 50% hexanes, 50% ethyl acetate, (iii) 100% ethyl acetate, (iv) 75% ethyl acetate, 25% methanol, and (v) 50% ethyl acetate, 50% methanol;
d) Drying flash chromatography fraction (iii) from (c);
e) Dissolving dried flash chromatography fraction (iii) from (e) in methanol: ethyl acetate (70:30) or 100% methanol;
f) Subjecting the dissolved fraction from (e) to preparative high performance liquid chromatography wherein a C18 preparative column is eluted with 30-70% acetonitrile and fractions are collected every minute;
g) Identifying the fraction or fractions from (f) containing the compound by an analytical technique;
h) Recovering the material contained in the fraction or fractions identified in (g) to obtain a pure preparation of the compound.

110. The process of claim 103, wherein the essentially pure compound is selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof.

111. The process of claim 110, wherein the essentially pure compound is selected from the group consisting of asiatic acid, madecassic acid, corosolic acid and salts, hydrates, solvates, prodrugs or N-oxides thereof.

112. The process of claim 111, wherein the essentially pure compound is corosolic acid or a salt, hydrate, solvate, prodrug or N-oxide thereof.

113. The process of claim 103, wherein the essentially pure compound comprises more than 1% by weight of the pharmaceutical composition.

114. The process of claim 103, wherein one and only one compound selected from the group consisting of ursolic acid, oleanolic acid, 30-hydroxyursolic acid, 2-hydroxyoleanolic acid, Corosolic acid, 3-O-[3-hydroxy, 4-methoxy-cinnamoyl(trans-)]-2hydroxyursolic acid, 3-[4-Hydroxycinnamoyl (cis-)], 20-hydroxy-ursolic acid, 3-[4-hydroxycinnamoyl(trans-)]-2-hydroxyursolic acid, 3-[4-hydroxycinnamoyl(cis-)]-2-hydroxyursolic acid, Euscaphic acid, 20β-hydroxyursolic acid, Tormentic acid, Asiatic acid, Madecassic acid, Pygenic acid A, Pygenic acid B, 3-hydroxy-12, 20(30) ursadienoic acid, Echinocystic acid, 3-acetyl oleanolic acid, Caulophyllogenin is obtained and combined to make the pharmaceutical composition.

115. The process of claim 114, wherein one and only one compound selected from the group consisting of 30-hydroxyursolic acid, 2-hydroxyoleanolic acid, corosolic acid, 3-O-[3-hydroxy, 4-methoxy-cinnamoyl(trans-)]-2-hydroxyursolic acid, asiatic acid, madecassic acid, and salts, hydrates, solvates, prodrugs and N-oxides thereof is obtained and combined to make the pharmaceutical composition.

116. The process of claim 115, wherein one and only one compound selected from the group consisting of corosolic acid, asiatic acid, madecassic acid, and salts, hydrates, solvates, prodrugs and N-oxides thereof is obtained and combined to make the pharmaceutical composition.

117. The process of claim 103, wherein less than 1.0% by weight triterpene glycoside, less than 1.0% by weight fatty acid, less than 1.0% by weight sterol, or less than 1.0% by weight hydrocarbon are found in the pharmaceutical composition.

118. The process of claim 103, wherein the essentially pure antimicrobial agent is selected from the group consisting of triclosan, metronidazole, tetracyclines, quinolones, plant essential oils, camphor, thymol, carvacrol, menthol, eucalyptol, methyl salicylate, tobramycin, clindamycin, ciprofloxacin, rifampin, oxfloxacin, macrolides, penicillins, cephalosporins, amoxicillin/clavulanate, quinupristin/dalfopristin, amoxicillin/sulbactum, fluoroquinolones, ketolides, and aminoglycosides.
